SEQUENCE LISTING (10 sequences)

SEQ ID NO: 1 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSTCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 2 - DNA, 942 bp, Homo sapiens
atggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccatgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagctggtgttattgctgttattgtggttgtggtgatagcagttgttgctggaattgttgtgctggttatttccagaaagaagagaatggcaaagtatgagaaggctgagataaaggagatgggtgagatgcatagggaactcaatgca

SEQ ID NO: 3 - DNA, 1528 bp, Homo sapiens
cggcgagcgagcaccttcgacgcggtccggggaccccctcgtcgctgtcctcccgacgcggacccgcgtgccccaggcctcgcgctgcccggccggctcctcgtgtcccactcccggcgcacgccctcccgcgagtcccgggcccctcccgcgcccctcttctcggcgcgcgcgcagcatggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccacgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagctggtgttattgctgttattgtggttgtggtgatagcagttgttgctggaattgttgtgctggttatttccagaaagaagagaatggcaaagtatgagaaggctgagataaaggagatgggtgagatgcatagggaactcaatgcataactatataatttgaagattatagaagaagggaaatagcaaatggacacaaattacaaatgtgtgtgcgtgggacgaagacatctttgaaggtcatgagtttgttagtttaacatcatatatttgtaatagtgaaacctgtactcaaaatataagcagcttgaaactggctttaccaatcttgaaatttgaccacaagtgtcttatatatgcagatctaatgtaaaatccagaacttggactccatcgttaaaattatttatgtgtaacattcaaatgtgtgcattaaatatgcttccacagtaaaatctgaaaaactgatttgtgattgaaagctgcctttctatttacttgagtcttgtacatacatacttttttatgagctatgaaataaaacattttaaactg

SEQ ID NO: 4 - DNA, 78 bp, Homo sapiens
tccagaaagaagagaatggcaaagtatgagaaggctgagataaaggagatgggtgagatgcatagggaactcaatgca

SEQ ID NO: 5 - DNA, 21 bp, Homo sapiens
aactacaagctggccgtaaac

SEQ ID NO: 6 - DNA, 20 bp, Homo sapiens
gctggtgtgtgaacactgct

SEQ ID NO: 7 - DNA, 34 bp, Homo sapiens
agaaggagatcacaacgcgttatcaactggatcc

SEQ ID NO: 8 - DNA, 21 bp, Homo sapiens
aacgcgttatcaactggatcc

SEQ ID NO: 9 - DNA, 20 bp, Homo sapiens
aaggagatgggtgagatgca

SEQ ID NO: 10 - DNA, 21 bp, Homo sapiens
aatccagaacttggactccat

(19) United States
(12) Patent Application Publication
(10) Pub. No.: US 2015/0094224 A1
(43) Pub. Date: Apr. 2, 2015

(54) METHODS FOR THE DIAGNOSIS OR PROGNOSIS OF BREAST CANCER

(71) Applicants: Paul WALFISH, Toronto (CA); Ranju RALHAN, Thornhill (CA)

(72) Inventors: Paul WALFISH, Toronto (CA); Ranju RALHAN, Thornhill (CA)

(21) Appl. No.: 14/501,020

(22) Filed: Sep. 29, 2014

Related U.S. Application Data
(63) Continuation-in-part of application No. 14/009,529, filed on Dec. 9, 2013.
Application No. 14/009,529 is a continuation-in-part of application No. 13/100,949, filed on May 4, 2011.
(60) Provisional application No. 61/332,358, filed on May 7, 2010.
(60) Provisional application No. 61/330,966, filed on May 4, 2010.

(51) Int. Cl. G01N 33/574; C12Q 1/68

(52) U.S. Cl. CPC G01N 33/57415; C12Q 1/6886; C12Q 2600/118; C12Q 2600/158; G01N 2800/52
USPC 506/9; 435/7.1; 435/6.12; 435/6.11; 435/6.14; 435/7.92

(57) ABSTRACT

Methods for detecting, diagnosing and monitoring an epithelial cancer in a patient are described comprising measuring in a sample from the patient Ep-ICD polypeptides and Ep-ICD polynucleotides. Methods for prognosis of breast cancer comprising measurement of nuclear Ep-ICD polypeptides and optionally EpEx polypeptides are provided. The invention also provides kits and compositions for carrying out the methods of the invention. The invention also provides a unique scoring system using immunohistochemical analysis to arrive at an Ep-ICD Subcellular Localization Index (ESLI) score, which is used to arrive at a diagnosis of cancer in a patient, or, more particularly, to identify patients that are in need of aggressive clinical treatment.

### CROSS REFERENCE TO RELATED APPLICATIONS

This application is a Continuation in Part of U.S. patent application Ser. No. 14/099,529, which is a Continuation in Part of U.S. patent application Ser. No. 13/100,949, filed May 4, 2011, which claims the benefit of priority under 35 U.S.C. §119(e) of U.S. Provisional Patent Application No. 61/330,966, filed May 4, 2010 and U.S. Provisional Patent Application No. 61/332,358, filed May 7, 2010. Each of the aforementioned applications is incorporated by reference herein as if set forth in its entirety.

### FIELD OF THE INVENTION

The invention relates to compositions, kits, and methods for detecting, diagnosing, monitoring, and characterizing epithelial cancers. The invention also relates to compositions, kits and methods for prognosis of breast cancer.

### BACKGROUND OF THE INVENTION

Breast cancer is the most frequently diagnosed cancer in females, with an estimated 1.38 million new cases per year worldwide and an estimated 226, 870 new cases in the United States in 2012 [Siegel et al., CA Cancer J. Clin. 2012, 62(1):10-29; and Ferlay et al., Int. J Cancer 2010, 127(12):2893-2917]. In early stage breast carcinoma patients, the presence of metastases to axillary lymph nodes is the most important predictor of survival [Fitzgibbons et al., Arch Pathol Lab Med 2000, 124(7):966-978]. Patients with node-positive tumors have up to an 8-fold increase in mortality than node-negative patients [Arriagada et al., Cancer 2006, 106(4):743-750]. The heterogenic nature of breast carcinomas and diverse patterns of growth and invasiveness stress the need for prognostic markers and predictive biological markers for aggressive tumors, particularly because many detected carcinomas may be non-aggressive [Zahl et al., The Lancet Oncology 2011, 12(12):1118-1124]. Population breast cancer screening with mammography may facilitate early detection of breast tumors and have the potential to lower mortality, but it is also associated with unnecessary treatment of tumors that would not have adversely affected the patient [Gotzsche & Jorgensen, Cochrane Database Syst Rev 2013, 6:CD001877]. Therefore, identifying aggressive lesions at an early stage is desirable.

Current clinical therapies for breast cancer include surgery, radiotherapy and drug therapies targeting oncogenic processes. Prediction of treatment response and propensity for metastasis is challenging, at least in part due to an incomplete understanding of the biology of various breast cancer subtypes. Many patients are over-treated to improve overall survival rates in early breast cancer. However, defining individual risk of disease recurrence and/or individual sensitivity to treatment would reduce over-treatment. Genomic tests (Mammaprint, Oncotype Dx, PAM50) and immunohistochemical tests (IHC 4) have been developed for prediction of breast cancer prognosis and response to chemotherapy but prospective validation of these is not currently available [Azim et al., Annals of Oncology 2013, 24(3):647-654]. Nuclear magnetic resonance (NMR) and mass spectrometry (MS)-based serum metabolite profiling has been shown to accurately identify 80% of breast cancer patients whose tumors failed to respond to chemotherapy suggesting promise for personalized treatment protocols [Wei et al., Molecular oncology 2013, 7(3):297-307]. Recently, a five-gene Integrated Cytokine score (ICS) has been proposed for predicting metastatic outcome from primary HRneg/Tneg breast tumors independent of nodal status, adjuvant chemotherapy use, and Tneg molecular subtype [Yau et al., Breast Cancer Research 2013, 15(5):R103]. Epithelial cell adhesion molecule (EpCAM) is a 40 kDa transmembrane glycoprotein that serves important roles in cell adhesion, cell proliferation, differentiation, migration, cell cycle regulation and is implicated in cancer and stem cell signaling [Munz et al., Cancer Res. 2009; 69:5627-5629]. EpCAM has been widely studied for its diagnostic and therapeutic potential as it is expressed in the majority of human epithelial cancers, including breast, colon, gastric, head and neck, prostate, pancreas, ovarian and lung cancer [Spizzo et al., Breast Cancer Research and Treatment 2004, 86(3):207-213; Went et al., Human Pathology 2004, 35(1):122-128; Saadatmand et al., The British Journal of Surgery 2013, 100(2):252-260; and Soysal et al., British Journal of Cancer 2013, 108(7):1480-1487]. Increased EpCAM expression has been found to be a marker of a poor prognosis in breast and gallbladder carcinomas. However, the prognostic relevance of Ep-ICD in these cancers has not been determined. Further, increased EpCAM expression does not necessarily reflect increased Ep-ICD in these cancers. [Gastl et al., Lancet 2000, 356(9246):1981-1982; and Varga et al., Clinical Cancer Research 2004, 10(9):3131-3136]. In contrast EpCAM expression in colorectal and gastric cancer is associated with favorable prognosis [Songun et al., British Journal of Cancer 2005, 92(9):1767-1772; and Went et al., British Journal of Cancer 2006, 94(1):128-135]. This paradoxical association between EpCAM expression and prognosis of various cancers is supported by functional studies of EpCAM biology using in vitro and in vivo cancer models as well (Schmidt et al., Ann Oncol 2010, 21(2):275-282). Together, these studies suggest that the pattern of EpCAM expression in human cancers is likely to be context dependent [van der Gun et al., Carcinogenesis 2010, 31(11):1913-1921].

An EpCAM expression-based assay has been FDA-approved and widely used to detect circulating tumor cells in breast cancer [Cristofanilli et al., The New England Journal of Medicine 2004, 351(8):781-791]. EpCAM has been widely explored as a potential target for antibody-based immunotherapies due to its high-expression and association with poor prognosis. EpCAM-targeted molecular therapies are being pursued for several cancers including breast, ovarian, gastric and lung cancer [Baeuerle et al., Br J Cancer 2007, 96(3):417-423]. EpCAM expression has been used to predict response to anti-EpCAM antibodies in breast cancer patients [Baeuerle & Gires, 2007; Schmidt et al., Ann Oncol 2012, 23(9):2306-2313; and Schmidt et al., Ann Oncol 2010, 21(2):275-282]. However, clinical trials of anti-EpCAM antibodies targeting the extracellular domain of EpCAM have shown limited efficacy [Schmidt et al., Ann Oncol 2010, 21(2):275-282; and Fields et al., J Clin Oncol 2009, 27(12):1941-1947].

These paradoxical outcomes might be due, at least in part, to the regulated intramembrane proteolysis of EpCAM which results in oncogenic signaling by the intracellular domain of EpCAM, Ep-ICD [Maetzel et al., Nat Cell Biol. 2009, 11:162-171]. Cleavage and shedding of the EpCAM ectodomain, EpEx, by proteases-TACE and Presenilin-2, releases the intracellular domain of EpCAM (Ep-ICD), which translocates to the nucleus. The association of Ep-ICD with FHL2 and Wnt pathway components, β-catenin and Lef-1, forms a nuclear complex that binds DNA at Lef-1 consensus sites and induces gene transcription, leading to increased cell proliferation and has been shown to be oncogenic in immunodeficient mice [Maetzel, 2009]. In view of the multiple roles of EpCAM as an oncogenic signal transducer, cell adhesion molecule and cancer stem cell marker [Litvinov et al., J Cell Biol. 1997; 139:1337-1348; and Munz et al., 2009], it is desirable to establish the clinical significance of Ep-ICD in human cancers.

A preliminary study reported expression of nuclear Ep-ICD in human colon cancer tissue, but not in the normal colonic epithelium [Maetzel et al, 2009]. However, in view of the tremendous heterogeneity in solid tumors, the clinical significance of nuclear Ep-ICD in other human cancers remains to be established.

Molecular markers for prognosis of cancer, in particular breast cancer, are desirable.

### SUMMARY OF THE DISCLOSURE

The present invention relates to biomarkers (i.e. Ep-ICD polypeptides and Ep-ICD polynucleotides hereinafter collectively referred to as “Epithelial Cancer Markers”), and agents that interact with the biomarkers, for detecting, diagnosing, characterizing, and monitoring epithelial cancer (e.g., monitoring progression of the cancer or the effectiveness of a therapeutic treatment), identifying subjects with a predisposition to epithelial cancer, and determining patient survival. In aspects of the invention, the Epithelial Cancer Markers are used in characterizing the aggressiveness of an epithelial cancer. In some aspects of the invention, the Epithelial Cancer Markers are used to determine metastatic potential or patient survival.

A method of the invention wherein Epithelial Cancer Marker(s) are assayed can have enhanced sensitivity and/or specificity relative to a method assaying other markers. The enhanced clinical sensitivity may be about a 5-10% increase, in particular 6-9% increase, more particularly 8% increase in sensitivity. In an embodiment, a method of the invention provides an epithelial cancer clinical sensitivity of at least about 80 to 99%, in particular 90 to 95%, more particularly 91%, 92%, 93%, or 94% epithelial cancer clinical sensitivity. In embodiments of the invention where Ep-ICD is detected in a tumor sample the clinical sensitivity can be greater than about 80 to 90%, more particularly greater than about 80 to 85%, most particularly greater than about 83%, 84%, or 85%. Clinical sensitivity and specificity may be determined using methods known to persons skilled in the art.

In accordance with methods of the invention, an Epithelial Cancer Marker in a sample may be assessed by detecting the presence in the sample of (a) a polypeptide or polypeptide fragment corresponding to the marker; (b) a transcribed nucleic acid or fragment thereof having at least a portion with which the marker is substantially identical; and/or (c) a transcribed nucleic acid or fragment thereof, wherein the nucleic acid hybridizes with the marker.

In an aspect of the invention, a method is provided for detecting Epithelial Cancer Markers associated with epithelial cancer in a patient comprising or consisting essentially of:
(a) obtaining a sample from a patient;
(b) detecting or identifying in the sample one or more Epithelial Cancer Markers and
(c) comparing the detected amount with an amount detected for a standard.

In an aspect, the invention provides a method for diagnosing an epithelial cancer in a subject, the method comprising:
(a) contacting a sample from a subject with a reagent capable of measuring a level of a target Epithelial Cancer Marker; and
(b) providing a diagnosis of an epithelial cancer in said subject based on an increase in the level of an Epithelial Cancer Marker in the sample from the subject over a control level obtained from similar samples taken from subjects who do not have the epithelial cancer or from the subject at a different time.

In an embodiment of the invention, a method is provided for diagnosing an epithelial cancer in a patient comprising or consisting essentially of:
(a) detecting or identifying in the sample Epithelial Cancer Markers; and
(b) comparing the detected amount with an amount detected for a standard, wherein an increase in Epithelial Cancer Markers is diagnostic of the epithelial cancer.

The invention provides a method for diagnosing an epithelial cancer in a subject comprising:
(a) detecting a level of an Ep-ICD polypeptide or a polynucleotide encoding an Ep-ICD polypeptide in a sample from the subject; and
(b) comparing the level detected in the subject's sample to levels detected for a predetermined standard.

In an aspect, the predetermined standard is a control level obtained from samples of the same type from subjects who do not have epithelial cancer; wherein an increased level of Ep-ICD polypeptide or polynucleotide encoding an Ep-ICD polypeptide in the sample from the subject over that of the control level is indicative of epithelial cancer.

The invention also provides a method for diagnosing an increased risk of an epithelial cancer, in a subject, the method comprising a) contacting a first sample from a subject at a first time with a diagnostic reagent that measures a first level of an Ep-ICD polypeptide; and b) diagnosing an increased risk of an epithelial cancer in the subject based upon an increased level of Ep-ICD polypeptide in the sample from the subject over that of (i) a first control level of Ep-ICD polypeptide obtained from samples of the same type taken from subjects who do not have the epithelial cancer; or (ii) an earlier sample level of Ep-ICD polypeptide obtained from samples of the same type taken from the same subject at an earlier time. In an aspect, a subject does not have an increased risk of developing an epithelial cancer if the first level is the same as either the first control level or the earlier sample level.

In a particular embodiment of the invention, a method is provided for diagnosing an epithelial cancer in a patient comprising or consisting essentially of:
(a) detecting or identifying in the sample Epithelial Cancer Markers and optionally EpEx (e.g. membranous EpEx), and
(b) comparing the detected amount with an amount detected for a standard, wherein an increase in Epithelial Cancer Markers and optionally a decrease or absence of EpEx is diagnostic of the epithelial cancer.

In a particular aspect of the invention, a method is provided for detecting Epithelial Cancer Markers in a patient comprising or consisting essentially of:
(a) obtaining a sample (e.g. tumor sample) from a patient;
(b) detecting in the sample Epithelial Cancer Markers; and
(c) comparing the detected amount with an amount detected for a standard or cut-off value.

The term “detect” or “detecting” includes assaying, or otherwise establishing the presence or absence of the target marker(s), subunits, or combinations of reagent bound targets, and the like, or assaying for ascertaining, establishing, characterizing, predicting or otherwise determining one or more factual characteristics of an epithelial cancer such as stage, aggressiveness, metastatic potential or patient survival, or assisting with same. A standard may correspond to levels quantitated for samples from control subjects with no disease or early stage disease (e.g., low grade epithelial cancer) or from other samples of the subject.

The invention provides a method of assessing whether a patient is at risk or afflicted with an epithelial cancer, the method comprising comparing:
(a) levels of Epithelial Cancer Markers from the patient; and
(b) standard levels of Epithelial Cancer Markers in samples of the same type obtained from control patients not afflicted with the epithelial cancer or with a lower grade of the epithelial cancer, wherein altered levels of Epithelial Cancer Markers relative to the corresponding standard levels of Epithelial Cancer Markers is an indication that the patient is at risk of or afflicted with the epithelial cancer.

In an aspect of a method of the invention for assessing whether a patient is at risk of or afflicted with an epithelial cancer, higher levels of Epithelial Cancer Markers, in a sample relative to corresponding normal levels or levels from a patient with a lower grade of epithelial cancer, is an indication that the patient is at risk of or afflicted with epithelial cancer.

In an embodiment of a method of the invention for assessing whether a patient is risk of or afflicted with epithelial cancer, levels of Epithelial Cancer Markers in a sample from the patient are compared to a standard, and higher levels of Epithelial Cancer Markers compared to a standard are indicative of epithelial cancer.

In an embodiment of a method of the invention for diagnosing breast cancer, levels of Epithelial Cancer Markers in a sample from the patient are compared to a standard.

In an embodiment of a method of the invention for diagnosing prostate cancer, levels of Epithelial Cancer Markers in a sample from the patient are compared to a standard.

In an embodiment of a method of the invention for diagnosing lung cancer, levels of Epithelial Cancer Markers in a sample from the patient are compared to a standard.

In an embodiment of a method of the invention for diagnosing pancreatic cancer, levels of Epithelial Cancer Markers in a sample from the patient are compared to a standard.

In an embodiment of a method of the invention for diagnosing urinary bladder cancer, levels of Epithelial Cancer Markers in a sample from the patient are compared to a standard.

In an embodiment of a method of the invention for diagnosing ovarian cancer, levels of Epithelial Cancer Markers in a sample from the patient are compared to a standard.

In an embodiment of a method of the invention for diagnosing liver cancer, levels of Epithelial Cancer Markers in a sample from the patient are compared to a standard.

In an embodiment of a method of the invention for diagnosing head and neck, levels of Epithelial Cancer Markers in a sample from the patient are compared to a standard.

In an embodiment of a method of the invention for diagnosing esophageal cancer, levels of Epithelial Cancer Markers in a sample from the patient are compared to a standard.

In particular aspects, methods of the invention are used to diagnose the stage of an epithelial cancer in a subject or characterizing epithelial cancer in a subject. In an embodiment, the method comprises comparing
(a) levels of a Epithelial Cancer Marker from a sample from the patient; and
(b) levels of the Epithelial Cancer Marker in control samples of the same type obtained from patients without epithelial cancer or control patients with a different stage of epithelial cancer, wherein altered levels of the Epithelial Cancer Marker, relative to the corresponding levels in the control samples is an indication that the patient is afflicted with a more aggressive or metastatic epithelial cancer.

The invention further provides a non-invasive non-surgical method for detection or diagnosis of epithelial cancer in a subject comprising: obtaining a sample (e.g., biopsy sample) from the subject; subjecting the sample to a procedure to detect Epithelial Cancer Marker(s); detecting or diagnosing epithelial cancer by comparing the levels of Epithelial Cancer Marker(s) to the levels of Epithelial Cancer Marker(s) obtained from a control subject with no epithelial cancer or a lower grade of epithelial cancer.

In an aspect, the invention provides a method for monitoring the progression of an epithelial cancer in a patient the method comprising:
(a) detecting Epithelial Cancer Marker(s) in a patient sample (e.g. biopsy sample) at a first time point;
(b) repeating step (a) at a subsequent point in time; and
(c) comparing the levels detected in (a) and (b), and thereby monitoring the progression of the epithelial cancer in the patient.

The invention provides a method for classifying a patient having epithelial cancer, the method comprising measuring Epithelial Cancer Marker(s) in a sample from the patient and correlating the values measured to values measured for the Epithelial Cancer Markers from epithelial cancer patients stratified in classification groups. The method can be used to predict patient survival, wherein the Epithelial Cancer Marker(s) are predictive of survival and wherein the classification groups comprise groups of known overall survival. In various embodiments the values measured can be normalized to provide more accurate quantification and to correct for experimental variations.

In particularly useful aspects of the invention, the Epithelial Cancer Markers detected are polynucleotides (“Ep-ICD polynucleotides”) and levels of Ep-ICD polynucleotides in a sample (e.g., biopsy sample) from a patient are compared with Ep-ICD polynucleotides levels from samples of patients without epithelial cancer, with a lower grade of epithelial cancer, or from levels from samples of the same patient. A method of the invention may employ one or more polynucleotides, oligonucleotides, or nucleic acids capable of hybridizing to Ep-ICD polynucleotides. In an aspect of the invention, Ep-ICD mRNA is detected.

The present invention relates to a method for diagnosing and characterizing epithelial cancer, more particularly the stage of epithelial cancer, in a sample from a subject comprising isolating nucleic acids, preferably mRNA, from the sample, and detecting Ep-ICD polynucleotides in the sample. In an embodiment, the presence of increased levels of Ep-ICD polynucleotides in the sample compared to a standard or control is indicative of epithelial cancer.

The invention also provides methods for determining the presence or absence of an epithelial cancer or the aggressiveness or metastatic potential of an epithelial cancer in a subject comprising detecting in the sample a level of nucleic acids that hybridize to an Ep-ICD polynucleotide, and comparing the level(s) with a predetermined standard or cut-off value, and therefrom determining the presence or absence of epithelial cancer or the aggressiveness or metastatic potential of an epithelial cancer in the subject. In an embodiment a method is provided for determining the aggressiveness or metastatic potential of epithelial cancer in a subject comprising (a) contacting a sample taken from the subject with oligonucleotides that hybridize to Ep-ICD polynucleotides; and (b) detecting in the sample a level of nucleic acids that hybridize to the oligonucleotides relative to a predetermined standard or cut-off value, and therefrom determining the aggressiveness or metastatic potential of the cancer in the subject.

In an aspect, the invention provides a method of assessing the aggressiveness or metastatic potential of an epithelial cancer in a patient, the method comprising comparing:
(a) levels of Ep-ICD polynucleotides in a sample from the patient; and
(b) control levels of Ep-ICD polynucleotides in samples of the same type obtained from control patients not afflicted with epithelial cancer or a lower grade of epithelial cancer, wherein altered levels of Ep-ICD polynucleotides relative to the corresponding control levels of Ep-ICD polynucleotides is an indication of the aggressiveness or metastatic potential of the epithelial cancer.

In a particular method of the invention for assessing whether a patient is afflicted with an aggressive or metastatic epithelial cancer, higher levels of Ep-ICD polynucleotides in a sample relative to the corresponding control levels is an indication that the patient is afflicted with an aggressive or metastatic epithelial cancer.

In an aspect, the invention provides a method for monitoring the progression of epithelial cancer in a patient, the method comprising:
(a) detecting Ep-ICD polynucleotides in a patient sample at a first time point; and
(b) repeating step (a) at a subsequent point in time; and
(c) comparing the levels detected in (a) and (b), and thereby monitoring the progression of epithelial cancer in the patient.

The invention further relates to a method of assessing the efficacy of a therapy for epithelial cancer in a patient. This method comprises comparing:
(a) levels of Ep-ICD polynucleotides in a first sample obtained from the patient prior to providing at least a portion of the therapy to the patient; and
(b) levels of Ep-ICD polynucleotides in a second sample obtained from the patient following therapy.

Significantly different levels of Ep-ICD polynucleotides in the second sample, relative to the first sample, can be an indication that the therapy is efficacious for inhibiting epithelial cancer. In an embodiment, the method is used to assess the efficacy of a therapy for inhibiting epithelial cancer, more particularly aggressive or metastatic epithelial cancer, and lower levels of Ep-ICD polynucleotides in the second sample relative to the first sample, is an indication that the therapy is efficacious for inhibiting the cancer or metastasis. The therapy may be any therapy for treating epithelial cancer including but not limited to chemotherapy, immunotherapy, gene therapy, radiation therapy, and surgical removal of tissue. Therefore, the method can be used to evaluate a patient before, during, and after therapy, for example, to evaluate the reduction in tumor burden, aggressiveness or metastatic potential of the tumor.

Within certain embodiments, the amount of nucleic acid that is mRNA is detected via amplification reactions such as polymerase chain reaction (PCR) using, for example, at least one oligonucleotide primer that hybridizes to an Ep-ICD polynucleotide or a complement of such polynucleotide. Within other embodiments, the amount of mRNA is detected using a hybridization technique, employing an oligonucleotide probe that hybridizes to an Ep-ICD polynucleotide, or a complement thereof.

When using mRNA detection, the method may be carried out by combining isolated mRNA with reagents to convert to cDNA according to standard methods; treating the converted cDNA with amplification reaction reagents along with an appropriate mixture of primers to produce amplification products; and analyzing the amplification products to detect the presence of Ep-ICD polynucleotides in the sample. For mRNA the analyzing step may be accomplished using RT-PCR analysis to detect the presence of Ep-ICD polynucleotides. The analysis step may be accomplished by quantitatively detecting the presence of Ep-ICD polynucleotides in the amplification product, and comparing the quantity of Ep-ICD polynucleotides, detected against a panel of expected values for known presence or absence in normal and malignant tissue (e.g., tissue from patients with a different stage of epithelial cancer), derived using similar primers.

Therefore, the invention provides a method wherein mRNA is detected by (a) isolating mRNA from a sample and combining the mRNA with reagents to convert it to cDNA; (b) treating the converted cDNA with amplification reaction reagents and nucleic acid primers that hybridize to an Ep-ICD polynucleotide to produce amplification products; (c) analyzing the amplification products to detect an amount of mRNA Ep-ICD polynucleotide; and (d) comparing the amount of mRNA to an amount detected against a panel of expected values for normal tissue and malignant tissue (e.g., tissue from patients with a different stage of epithelial cancer) derived using similar nucleic acid primers.

Protein based methods can also be used for diagnosing and monitoring epithelial cancer, in particular the aggressiveness or metastatic potential of epithelial cancer in a subject comprising detecting Ep-ICD polypeptides in a sample from the subject. Ep-ICD polypeptides may be detected using a binding agent for Ep-ICD polypeptides, preferably antibodies specifically reactive with Ep-ICD polypeptides.

The invention provides a method of assessing whether a patient is afflicted with or at risk of epithelial cancer which comprises comparing:
(a) levels of Ep-ICD polypeptides in a sample from the patient; and
(b) control levels of Ep-ICD polypeptides in a non-cancer sample or sample from a patient with a lower grade of epithelial cancer, wherein significantly different levels of Ep-ICD polypeptides in the sample from the patient compared with the control levels (e.g. higher in the patient samples) is an indication that the patient is afflicted with or at risk of epithelial cancer.

In another aspect the invention provides methods for determining the presence or absence of epithelial cancer or the aggressiveness or metastatic potential of a epithelial cancer in a patient comprising the steps of (a) contacting a biological sample obtained from a patient with a binding agent that specifically binds to an Ep-ICD polypeptide; and (b) detecting in the sample an amount of Ep-ICD polypeptide that binds to the binding agent(s), relative to a predetermined standard or cut-off value, and therefrom determining the presence or absence of the epithelial cancer or the aggressiveness or metastatic potential of the epithelial cancer in the patient.

In an embodiment, the invention relates to a method for detecting, diagnosing, staging and monitoring epithelial cancer in a subject by quantitating an Ep-ICD polypeptide in a biological sample from the subject comprising (a) reacting the biological sample with an antibody specific for the Ep-ICD polypeptide which is directly or indirectly labeled with a detectable substance; and (b) detecting the detectable substance.

In another embodiment the invention provides a method of using antibodies to detect expression of Ep-ICD polypeptides in a sample, the method comprising: (a) combining antibodies specific for Ep-ICD polypeptides with a sample under conditions which allow the formation of antibody:protein complexes; and (b) detecting complex formation, wherein complex formation indicates expression of Ep-ICD polypeptides in the sample. Expression may be compared with standards and is diagnostic of epithelial cancer or the aggressiveness or metastatic potential of the epithelial cancer.

In an aspect, the invention provides a method for monitoring the progression of epithelial cancer in a patient, the method comprising:
(a) detecting Ep-ICD polypeptides in a patient sample at a first time point;
(b) repeating step (a) at a subsequent point in time; and
(c) comparing the levels detected in (a) and (b), and thereby monitoring the progression of epithelial cancer in the patient.

The invention further relates to a method of assessing the efficacy of a therapy for epithelial cancer in a patient. This method comprises comparing:
(a) levels of Ep-ICD polypeptides in a first sample obtained from the patient prior to providing at least a portion of the therapy to the patient; and
(b) levels of Ep-ICD polypeptides in a second sample obtained from the patient following therapy.

Significantly different levels of Ep-ICD polypeptides in the second sample, relative to the first sample, can be an indication that the therapy is efficacious for inhibiting epithelial cancer. In an embodiment, the method is used to assess the efficacy of a therapy for inhibiting epithelial cancer, more particularly aggressive or metastatic epithelial cancer, and lower levels of Ep-ICD polypeptides in the second sample relative to the first sample, is an indication that the therapy is efficacious for inhibiting the cancer or metastasis. The therapy may be any therapy for treating epithelial cancer including but not limited to chemotherapy, immunotherapy, gene therapy, radiation therapy, and surgical removal of tissue. Therefore, the method can be used to evaluate a patient before, during, and after therapy, for example, to evaluate the reduction in tumor burden, aggressiveness or metastatic potential of the tumor.

The invention also provides a composition for diagnosing an epithelial cancer comprising Epithelial Cancer Markers or agents that interact with Epithelial Cancer Markers. In particular, the invention provides a composition for diagnosing an epithelial cancer comprising Ep-ICD polypeptides, or agents that bind to Ep-ICD polypeptides, or hybridize to or amplify Ep-ICD polynucleotides.

In an embodiment, the composition comprises a probe that specifically hybridizes to an Ep-ICD polynucleotide or a fragment thereof, and a probe that specifically hybridizes to a Ep-ICD polynucleotide or a fragment thereof. In another embodiment a composition is provided comprising a specific primer(s) pair capable of amplifying an Ep-ICD polynucleotide using polymerase chain reaction methodologies. In a still further embodiment, the composition comprises a binding agent(s) (e.g. antibody) that binds to an Ep-ICD polypeptide or a fragment thereof. Probes, primers, and binding agents can be labeled with a detectable substance.

In an embodiment, a diagnostic composition of the invention comprises antibodies specific for Ep-ICD polypeptides. In an embodiment, a diagnostic composition of the invention comprises primers that amplify Ep-ICD polynucleotides.

In another aspect, the invention relates to use of an agent that interacts with an Epithelial Cancer Marker in the manufacture of a composition for diagnosing epithelial cancer.

The methods of the invention may also comprise detecting additional markers associated with an epithelial cancer. In embodiments of the methods of the invention, EpCAM and membranous EpEx, and/or polynucleotides encoding same are detected.

Further, the amount of Epithelial Cancer Markers may be mathematically combined with other markers of epithelial cancer. In an embodiment the invention provides a method for detecting or diagnosing epithelial cancer in a subject comprising:
(a) determining the amount of Epithelial Cancer Markers in a sample from the subject;
(b) determining the amount of other markers associated with the epithelial cancer (e.g. EpCAM or EpEx);
(c) mathematically combining the results of step (a) and step (b) to provide a mathematical combination; and
(d) comparing or correlating the mathematical combination to the presence of epithelial cancer or aggressiveness or metastatic potential of epithelial cancer.

The combination is preferably compared to a mathematical combination for a predetermined standard.

The invention also includes kits for carrying out methods of the invention. In an aspect the invention provides a kit for detecting, diagnosing or characterizing an epithelial cancer comprising Epithelial Cancer Markers. In a particular aspect, the invention provides a test kit for diagnosing or characterizing epithelial cancer in a subject which comprises an agent that interacts with an Epithelial Cancer Marker(s). In an embodiment, the kit comprises reagents for identifying and/or assessing levels of nuclear Ep-ICD polypeptide.

The invention therefore contemplates an in vivo method comprising administering to a mammal one or more agent that carries a label for imaging and binds to an Epithelial Cancer Marker, and then imaging the mammal. According to a preferred aspect of the invention, an in vivo method for imaging epithelial cancer is provided comprising:
(a) injecting a patient with an agent that binds to an Epithelial Cancer Marker(s), the agent carrying a label for imaging the epithelial cancer;
(b) allowing the agent to incubate in vivo and bind to the Epithelial Cancer Marker(s); and
(c) detecting the presence of the label localized to the epithelial cancer.

In an embodiment of the invention the agent is an antibody which recognizes the Epithelial Cancer Marker(s). In another embodiment of the invention the agent is a chemical entity which recognizes the Epithelial Cancer Marker(s).

The agent carries a label to image the Epithelial Cancer Marker(s). Examples of labels useful for imaging are radiolabels, fluorescent labels (e.g., fluorescein and rhodamine), nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes, can also be employed.

The invention also contemplates the localization or imaging methods described herein using multiple markers for epithelial cancer.

In an aspect, the invention provides antagonists (e.g. antibodies) specific for an Epithelial Cancer Marker, in particular Ep-ICD that can be used therapeutically to destroy or inhibit the growth of epithelial cancer cells, or to block activity. In addition, Epithelial Cancer Markers may be used in various immunotherapeutic methods to promote immune-mediated destruction or growth inhibition of tumors expressing Epithelial Cancer Markers.

In another aspect, the invention provides a method for prognosing breast cancer in a subject. In some embodiments, the method comprises detecting a level of nuclear Ep-ICD in a biological sample from the subject, comparing the level detected in the biological sample to a control and prognosing breast cancer based on the comparison between the detected level of nuclear Ep-ICD and the control.

In some embodiments of this aspect of the invention, if the control is a level of nuclear Ep-ICD in a non-cancerous biological sample, then a higher detected level of nuclear Ep-ICD indicates a poor prognosis, and an equal or lower detected level of nuclear Ep-ICD indicates a favorable prognosis; if the control is the level of nuclear Ep-ICD in a biological sample known not to progress to breast cancer for at least 40 months following measurement of the control level, then a higher detected level of nuclear Ep-ICD indicates a poor prognosis, and an equal or lower detected level of nuclear Ep-ICD indicates a favorable prognosis; or if the control is the level of nuclear Ep-ICD in a biological sample known to progress to breast cancer in less than about five years following measurement of the control level, then an equal or higher detected level of nuclear Ep-ICD indicates a poor prognosis, and a lower detected level of nuclear Ep-ICD indicates a favorable prognosis.

In some embodiments, a poor prognosis comprises one or more of disease free survival of less than five years and overall survival of less than five years. In some embodiments, a poor prognosis comprises disease free survival of less than or equal to about 41 months.

In some embodiments, a favorable prognosis comprises one or more of disease free survival of at least about five years and overall survival of at least about five years.

In some embodiments of the method for prognosing breast cancer in a subject the sample from the subject comprises breast epithelial cells. In some embodiments, the sample comprises breast tissue. In some embodiments the sample comprises stage I or II breast cancer tumor cells.

In some embodiments of the method for prognosing breast cancer in a subject the breast cancer is invasive ductal carcinoma, invasive lobular carcinoma, invasive mucinous carcinoma, ductal carcinoma in situ or lobular carcinoma in situ.

In another aspect, the invention provides a method for establishing the prognosis of breast cancer in a subject. In some embodiments, the method comprises detecting the presence or absence of nuclear Ep-ICD in a biological sample from the subject, the biological sample comprising breast epithelial cells.

In another aspect, the invention provides a method for determining whether a subject has breast cancer with a poor prognosis. In some embodiments, the method comprises detecting the presence or absence of nuclear Ep-ICD in a biological sample from the subject, the biological sample comprising breast epithelial cells, the presence of nuclear Ep-ICD indicating that the subject has breast cancer associated with a poor prognosis.

In another aspect, the invention provides a method for the prognosis of survival time of a subject having breast cancer. In some embodiments, the method comprises detecting the level of nuclear Ep-ICD in a biological sample from the patient, comparing the detected level of nuclear Ep-ICD with one or more predetermined reference values correlated with a specific prognoses of survival time, and determining a prognosis for the subject. In some embodiments, a favorable prognosis of survival time is provided for the subject when the detected level of nuclear Ep-ICD is lower than said corresponding predetermined reference values. In some embodiments, a poor prognosis of survival time is provided for the subject when the detected value is lower than said corresponding predetermined cut-off reference values.

In some embodiments, the method for prognosing breast cancer in a subject further comprises detecting a level of EpEx in a sample from the subject and comparing the detected level of EpEx with a control.

In some embodiments, the method comprises detecting the presence or absence of membranous EpEx in a sample from the subject.

In some embodiments, the method comprises detecting loss of membranous EpEx in a sample from the subject.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1 shows immunohistochemical analysis of Ep-ICD in breast cancer and prostate cancer. Arrows show Ep-ICD nuclear staining. Original magnification ×40.

FIG. 2 shows immunohistochemical analysis of Ep-ICD in colon cancer, bladder cancer, ovarian cancer, lung cancer, liver cancer and pancreatic cancer. Original magnification ×40.

FIG. 3 is a scatter plot showing Ep-ICD membranous expression in different kinds of cancers.

FIG. 4 is a scatter plot showing cytoplasmic Ep-ICD expression in epithelial tissues and cancers.

FIG. 5 is a scatter plot showing nuclear Ep-ICD expression in epithelial tissues and cancers.

FIG. 6 is an ROC curve of Ep-ICD nuclear expression in breast cancer. AUC: 0.968. Sensitivity: 94.12. Specificity: 100. Criterion value: >1.7

FIG. 7 is an ROC curve of Ep-ICD nuclear expression in prostate cancer. AUC: 0.973. Sensitivity: 95.56. Specificity: 100. Criterion value: >2.67

FIG. 8 shows Ep-ICD and beta catenin immunostaining in head and neck cancer. Original magnification ×20.

FIG. 9 shows Ep-ICD immunostaining in head and neck cancer. Original magnification ×20.

FIGS. 10A to 10C show: a Box Plot of cytoplasmic Ep-ICD staining in head and neck/oral cancer (FIG. 10A) and nuclear Ep-ICD staining in head and neck/oral cancer (FIG. 10B); and an ROC curve of Ep-ICD cytoplasmic and nuclear expression in neck/oral cancer (FIG. 10C). FIG. 11 shows immunohistochemical analysis of Ep-ICD protein in esophageal tissues. Original magnification ×20.

FIGS. 12A to 12G show immunofluorescence analysis of Ep-ICD in breast cancer tissues, wherein Ep-ICD (red), EpEx (green) and nucleic DNA (blue) were monitored with specific antibodies and DAPI, respectively. FIG. 12A shows nucleic DNA stained with DAPI (blue); FIG. 12B shows subcellular localization of Ep-ICD (red) in breast cancer cells; FIG. 12C shows subcellular localization of EpEx (green) in breast cancer cells; FIG. 12D shows a merged image of FIGS. 12B and 12C showing Ep-ICD localized in cytoplasm and nuclei; FIG. 12E shows a merged image of FIGS. 12A and 12B showing the nuclear colocalization of Ep-ICD and DAPI; FIG. 12F shows a merged image of FIGS. 12A and 12C showing dominating DAPI nuclear staining; and FIG. 12G shows a merged image of FIGS. 12A, 12B and 12C reshowing the nuclear colocalization of Ep-ICD and DAPI and also the cytoplasmic Ep-ICD. The magnification is shown by the scale bar.

FIGS. 13A to 13G show immunofluorescence analysis of Ep-ICD in colon cancer tissues, wherein Ep-ICD (red), EpEx (green) and nucleic DNA (blue) were monitored with specific antibodies and DAPI, respectively in colon cancer paraffin section. FIG. 13A shows nucleic DNA stained with DAPI (blue) in colon cancer cells; FIG. 13B shows subcellular localization of Ep-ICD (red) in colon cancer cells; FIG. 13C shows subcellular localization of EpEx (green) in colon cancer; FIG. 13D shows a merged image of FIGS. 13A and 13C showing dominating DAPI nuclear staining and strong EpEx cytoplasmic staining; FIG. 13E shows a merged image of FIGS. 13A and 13B showing the nuclear colocalization of Ep-ICD and DAPI; FIG. 13F shows a merged image of FIGS. 13B and 13C showing Ep-ICD colocalized with EpEx in cytoplasm and also in nuclei; and FIG. 13G shows a merged image of FIGS. 13A, 13B and 13C showing the nuclear colocalization of Ep-ICD and nuclear DNA and also showing cytoplasmic Ep-ICD and EpEx in colon cancer cells. The magnification is shown by the scale bar.

FIGS. 14A to 14G show immunofluorescence analysis of Ep-ICD in prostate cancer tissuespp, wherein subcellular localization of Ep-ICD (red), EpEx (green) and nucleic DNA (blue) were observed with specific antibodies and DAPI, respectively in human prostate cancer paraffin section. FIG. 14A shows nucleic DNA stained with DAPI (blue) in prostate cancer cells; FIG. 14B shows subcellular localization of Ep-ICD (red) in prostate cancer cells; FIG. 14C shows subcellular localization of EpEx (green) in prostate cancer cells; FIG. 14D shows a merged image of FIGS. 14A and 14C showing DAPI nuclear DNA staining, weak EpEx membrane staining and strong cytoplasmic staining in prostate cancer; FIG. 14E shows a merged image of FIGS. 14A and 14B showing the nuclear colocalization of Ep-ICD and DAPI; FIG. 14F shows a merged image of FIGS. 14B and 14C showing Ep-ICD colocalized with EpEx in cytoplasm; and FIG. 14G shows a merged image of FIGS. 14A, 14B and 14C showing the nuclear colocalization of Ep-ICD and nuclear DNA and also showing the cytoplasmic Ep-ICD and EpEx in prostate cancer cells. The magnification is shown by the scale bar.

FIG. 15 is a nucleic acid sequence encoding an Ep-ICD Polypeptide showing siRNA targets [SEQ ID NOs. 3 and 5-10].

FIGS. 16A and 16B depict immunohistochemical analysis of Ep-ICD and EpEx expression in breast cancer. (A) Representative photomicrographs demonstrating: (I) predominantly cytoplasmic Ep-ICD expression in normal breast tissues. Nuclear and cytoplasmic accumulation of Ep-ICD in: (II) DCIS; (III) IDC; (IV) ILC; (V) IMC; and (VI) negative control breast cancer tissue incubated with isotype specific IgG showing no detectable immunostaining for Ep-ICD. (B) Expression of EpEx in (I) normal breast tissues; (II) DCIS; (III) IDC; (IV) ILC; (V) IMC. Membranous EpEx expression was more frequently observed in breast carcinomas compared to normal tissues, except ILC (original magnification ×400). The arrows labelled N, C and M depict nuclear, cytoplasmic and membrane staining respectively.

FIGS. 17A and 17B depict Kaplan-Meier curves for disease-free survival (DFS) stratified by nuclear Ep-ICD expression in all breast carcinoma patients and in IDC. (A) Nuclear accumulation of Ep-ICD was associated with significantly reduced DFS in the entire cohort of breast carcinoma patients (p<0.001). (B) Nuclear accumulation of Ep-ICD was associated with significantly reduced DFS in IDC patients (p<0.001).

FIGS. 18A and 18B show the Ep-ICD Subcellular Localization Index (ESLI) scores for the results from Example 2.

### DETAILED DESCRIPTION OF THE DISCLOSURE

The invention relates to newly discovered correlations between expression of Epithelial Cancer Markers and epithelial cancers. The Epithelial Cancer Markers described herein provide methods for diagnosing, detecting or characterizing epithelial cancers. Methods are provided for diagnosing or detecting the presence or absence of an epithelial cancer in a sample, and for monitoring the progression of an epithelial cancer, as well as providing information about characteristics of an epithelial cancer that are relevant to the diagnosis and characterization of an epithelial cancer in a patient.

### GLOSSARY

Unless defined otherwise, all technical and scientific terms used herein have the same meaning as commonly understood by one of ordinary skill in the art to which this invention belongs. The following definitions supplement those in the art and are directed to the present application and are not to be imputed to any related or unrelated case. Although any methods and materials similar or equivalent to those described herein can be used in the practice of the invention, particular materials and methods are described herein.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. As used herein and in the appended claims, the singular forms “a”, “an”, and “the” include plural reference unless the context clearly dictates otherwise.

The term “epithelial cancer” refers to any malignant process that has an epithelial origin. Examples of epithelial cancers include, but are not limited to, a gynecological cancer such as endometrial cancer, ovarian cancer, cervical cancer, vulvar cancer, uterine cancer or fallopian tube cancer, breast cancer, prostate cancer, lung cancer, pancreatic cancer, urinary cancer, bladder cancer, head and neck cancer, oral cancer and liver cancer. An epithelial cancer may be at different stages as well as varying degrees of grading. In embodiments, the epithelial cancer is selected from the group consisting of breast cancer, prostate cancer, lung cancer, pancreatic cancer, bladder cancer and ovarian cancer. In a particular embodiment, the epithelial cancer is breast cancer. In a particular embodiment, the epithelial cancer is ovarian cancer. In a particular embodiment, the epithelial cancer is prostate cancer. In a particular embodiment, the epithelial cancer is lung cancer. In a particular embodiment, the epithelial cancer is head and neck cancer. In a particular embodiment, the epithelial cancer is head and neck squamous cell carcinoma.

“Metastatic potential” refers to the ability or possibility of a cancer cell moving from the initial site to other sites in the body.

The term “sample” and the like mean a material known or suspected of expressing or containing Epithelial Cancer Markers, or binding agents such as antibodies specific for Ep-ICD polypeptides. The sample may be derived from a biological source (“biological sample”), such as tissues, extracts, or cell cultures, including cells (e.g. tumor cells), cell lysates, and biological or physiological fluids, such as, for example, whole blood, plasma, serum, saliva, cerebral spinal fluid, sweat, urine, milk, peritoneal fluid and the like. A sample may be used directly as obtained from the source or following a pretreatment to modify the character of the sample, such as preparing plasma from blood, diluting viscous fluids, and the like. In certain aspects of the invention, the sample is a human physiological fluid, such as human serum. In certain aspects of the invention, the sample is a biopsy sample. In certain aspects of the invention the sample is a benign, malignant, or normal tissue sample.

The samples that may be analyzed in accordance with the invention include polynucleotides from clinically relevant sources, preferably expressed RNA or a nucleic acid derived therefrom (cDNA or amplified RNA derived from cDNA that incorporates an RNA polymerase promoter). As will be appreciated by those skilled in the art, the target polynucleotides can comprise RNA, including, without limitation total cellular RNA, poly(A) messenger RNA (mRNA) or fraction thereof, cytoplasmic mRNA, or RNA transcribed from cDNA (i.e., cRNA).

Target polynucleotides can be detectably labeled at one or more nucleotides using methods known in the art. The label is preferably uniformly incorporated along the length of the RNA, and more preferably, is carried out at a high degree of efficiency. The detectable label can be a luminescent label, fluorescent label, bio-luminescent label, chemi-luminescent label, radiolabel, and colorimetric label.

Target polynucleotides from a patient sample can be labeled differentially from polynucleotides of a standard. The standard can comprise target polynucleotides from normal individuals (e.g. those not afflicted with or pre-disposed to an epithelial cancer, in particular pooled from samples from normal individuals or patients with a different disease stage). The target polynucleotides can be derived from the same individual, but taken at different time points, and thus indicate the efficacy of a treatment by a change in expression of the markers, or lack thereof, during and after the course of treatment.

The terms “subject”, “patient” and “individual” are used interchangeably herein and refer to a warm-blooded animal such as a mammal that is afflicted with, or suspected of having, at risk for or being pre-disposed to, or being screened for epithelial cancer, in particular actual or suspected epithelial cancer. The term includes but is not limited to domestic animals, sports animals, primates and humans. Preferably, the terms refer to a human.

A subject suspected of having epithelial cancer includes a subject that presents one or more symptoms indicative of an epithelial cancer (e.g., a noticeable lump or mass) or is being screened for a cancer (e.g., during a routine physical). A subject suspected of having an epithelial cancer may also have one or more risk factors. A subject suspected of having epithelial cancer has generally not been tested for cancer. However, a subject suspected of having epithelial cancer encompasses an individual who has received an initial diagnosis but for whom the stage of cancer is not known and people who once had cancer (e.g., an individual in remission).

A subject at risk for or being pre-disposed to epithelial cancer includes a subject with one or more risk factors for developing an epithelial cancer. Risk factors include, but are not limited to, gender, age, genetic predisposition, environmental exposure, previous incidents of cancer, pre-existing non-cancer diseases, and lifestyle.

As used herein, the term “characterizing epithelial cancer in a subject” refers to the identification of one or more properties of a cancer sample in a subject, including but not limited to the subject's prognosis or survival. Cancers may be characterized by the identification of the expression of one or more markers, including but not limited to, the Epithelial Cancer Markers disclosed herein.

“Polypeptide” and “protein” are used interchangeably herein and indicate at least one molecular chain of amino acids linked through covalent and/or non-covalent bonds. The terms include peptides, oligopeptides, and proteins, and post-translational modifications of the polypeptides, e.g. glycosylations, acetylations, phosphorylations, and the like. Protein fragments, analogues, mutated or variant proteins, fusion proteins, and the like, are also included within the meaning of the terms.

The term “EpCAM” refers to a type I membrane protein comprising an epidermal growth factor (EGF)-like domain and a thyroglobulin repeat domain. In particular, it is composed of a large extracellular domain (265 amino acids) (EpEx), a single transmembrane part of 23 amino acids (e.g. amino acids 266-288 in SEQ ID NO. 1), and a short cytoplasmic domain of 26 amino acids (e.g. Ep-ICD, amino acids 289-314 in SEQ ID NO. 1). Two EGF-like repeats are located within the extracellular domain [Balzar et al., Mol Cell Biol. 2001 21(7):2570-80]. The mature enzyme consists of 314 amino acids. See Baeuerie P A and O Gires, British Journal of Cancer (2007) 96, pages 417-423 for a review of EpCAM (CD326).] The term includes native-sequence polypeptides, isoforms, polypeptide variants, precursors, and chimeric or fusion proteins of EpCAM, in particular human EpCAM. EpCAM polypeptides include, without limitation, polypeptides comprising the sequences found in Accession No. NP_{—}002345 and SEQ ID NO. 1.

“Epithelial Cancer Markers” includes “Ep-ICD polypeptides” and “Ep-ICD polynucleotides”.

The terms “Ep-ICD polypeptides” and “Ep-ICD” refer to a polypeptide comprising the cytoplasmic domain of EpCAM. The cytoplasmic domain of EpCAM comprises about 26 amino acids of EpCAM. The terms also include native-sequence polypeptides, isoforms, fragments, polypeptide variants and chimeric or fusion proteins thereof. In particular, the terms include the sequence comprising amino acids 289-314 found in Accession No. NP_{—}002345 and SEQ ID NO. 1. In embodiments of the invention, the Ep-ICD polypeptide is a Ep-ICD polypeptide associated with the nucleus.

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” means a polypeptide having at least about 10%, 20%, 30%, 40%, 45%, 50%, 55%, 60%, 65%, 70%, 75%, 80%, 85%, 90%, 95%, 97%, 98%, or 99% amino acid sequence identity, particularly at least about 70-80%, more particularly at least about 85%, still more particularly at least about 90%, most particularly at least about 95%, 97%, or 99% amino acid sequence identity with a native-sequence polypeptide. Particular polypeptide variants have at least 70-80%, 85%, 90%, 95%, 97% or 99% amino acid sequence identity to sequences identified in Accession No. NP_{—}002345 and SEQ ID NO: 1, in particular amino acids 289-314 found in Accession No. NP_{—}002345 and SEQ ID NO: 1. Such variants include, for instance, polypeptides wherein one or more amino acid residues are added to, or deleted from, the N- or C-terminus of the full-length or mature sequences of the polypeptide, including variants from other species, but exclude a native-sequence polypeptide. In aspects of the invention variants retain the immunogenic activity of the corresponding native-sequence polypeptide.

Sequence identity of two amino acid sequences or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

Polypeptide variants include polypeptides comprising amino acid sequences sufficiently identical to or derived from the amino acid sequence of a native polypeptide which includes fewer amino acids than the native polypeptides. A portion or fragment of a polypeptide can be a polypeptide which is for example, 3-5, 8-10, 10, 15, 15-20, 20, 25, 30, 35, 40, 45, 50, 60, 70, 80, 90, 100 or more amino acids in length. Portions or fragments in which regions of a polypeptide are deleted can be prepared by recombinant techniques and can be evaluated for one or more functional activities such as the ability to form antibodies specific for a polypeptide. A portion or fragment of a polypeptide may comprise a domain of the polypeptide or a portion or fragment of such domain.

An allelic variant may also be created by introducing substitutions, additions, or deletions into a nucleic acid encoding a native polypeptide sequence or domain thereof such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain, several of which are known in the art.

A naturally occurring allelic variant may contain conservative amino acid substitutions from the native polypeptide sequence or domain thereof, or it may contain a substitution of an amino acid from a corresponding position in polypeptide homolog, for example, a murine polypeptide.

A polypeptide disclosed herein includes chimeric or fusion proteins. A “chimeric protein” or “fusion protein” comprises all or part (preferably biologically active) of the polypeptide operably linked to a heterologous polypeptide (i.e., a different polypeptide). Within the fusion protein, the term “operably linked” is intended to indicate that the polypeptide and the heterologous polypeptide are fused in-frame to each other. The heterologous polypeptide can be fused to the N-terminus or C-terminus of the polypeptide. A useful fusion protein is a GST fusion protein in which a polypeptide is fused to the C-terminus of GST sequences. Another example of a fusion protein is an immunoglobulin fusion protein in which all or part of a polypeptide is fused to sequences derived from a member of the immunoglobulin protein family. Chimeric and fusion proteins can be produced by standard recombinant DNA techniques.

Polypeptides used in the methods disclosed herein may be isolated from a variety of sources, such as from human tissue types or from other sources, or prepared by recombinant or synthetic methods, or by any combination of these and similar techniques.

“Polynucleotide” refers to a polymeric form of nucleotides of any length, either ribonucleotides or deoxyribonucleotides. The term includes double- and single-stranded DNA and RNA, modifications such as methylation or capping and unmodified forms of the polynucleotide. The terms “polynucleotide” and “oligonucleotide” are used interchangeably herein. A polynucleotide may, but need not, include additional coding or non-coding sequences, or it may, but need not, be linked to other molecules and/or carrier or support materials. Polynucleotides for use in the methods of the invention may be of any length suitable for a particular method. In certain applications the term refers to antisense nucleic acid molecules (e.g. an mRNA or DNA strand in the reverse orientation to a sense Ep-ICD polynucleotide).

“Ep-ICD polynucleotides” include polynucleotides encoding an Ep-ICD polypeptide, including a native-sequence polypeptide, a polypeptide variant including a portion of an Ep-ICD polypeptide, an isoform, precursor, and a chimeric polypeptide. A polynucleotide encoding an EpCAM polypeptide that can be employed in the present invention includes, without limitation, nucleic acids comprising a sequence of Accession No. UniProtKB/TrEMBL Q6FG26 or SEQ ID NOs. 2 or 3 encoding an Ep-ICD Polypeptide. A polynucleotide encoding Ep-ICD that can be employed in the present invention includes, without limitation, nucleic acids comprising the sequence of Accession No. NM_{—}002354_{—}11 or SEQ ID NO. 4 encoding an Ep-ICD Polypeptide.

Polynucleotides used in the methods of the invention include complementary nucleic acid sequences, and nucleic acids that are substantially identical to these sequences (e.g. at least about 10%, 20%, 30%, 40%, or 45%, preferably 50%, 55%, 60%, 65%, 70%, 75%, 80%, 85%, 90%, 95%, 97%, 98%, or 99% sequence identity).

Polynucleotides also include sequences that differ from a nucleic acid sequence due to degeneracy in the genetic code. As one example, DNA sequence polymorphisms within the nucleotide sequence of an Epithelial Cancer Marker disclosed herein may result in silent mutations that do not affect the amino acid sequence. Variations in one or more nucleotides may exist among individuals within a population due to natural allelic variation. DNA sequence polymorphisms may also occur which lead to changes in the amino acid sequence of a polypeptide.

Polynucleotides which may be used in the methods disclosed herein also include nucleic acids that hybridize under stringent conditions, preferably high stringency conditions to a nucleic acid sequence of an Ep-ICD polynucleotide. Appropriate stringency conditions which promote DNA hybridization are known to those skilled in the art, or can be found in Ausubel et al., (eds) Current Protocols in Molecular Biology, John Wiley & Sons, N.Y. (1989), 6.3.1-6.3.6. Generally, stringent conditions may be selected that are about 5° C. lower than the thermal melting point (Tm) for the specific sequence at a defined ionic strength and pH. The Tm is the temperature (under defined ionic strength, pH, and nucleic acid concentration) at which 50% of the probes complementary to a target sequence hybridize at equilibrium to the target sequence. Generally, stringent conditions will be those in which the salt concentration is less than about 1.0M sodium ion or other salts (e.g. about 0.01 to 1.0M sodium ion) and the temperature is at least about 30° C. for short probes, primers or oligonucleotides (e.g. 10-50 nucleotides) and at least 60° C. for longer probes, primers and oligonucleotides. For example, a hybridization may be conducted at 6.0× sodium chloride/sodium citrate (SSC) at about 45° C., followed by a wash of 2.0×SSC at 50° C., or at 42° C. in a solution containing 6×SCC, 0.5% SDS and 50% formamide followed by washing in a solution of 0.1×SCC and 0.5% SDS at 68° C.

Ep-ICD polynucleotides also include truncated nucleic acids or nucleic acid fragments and variant forms of the nucleic acids disclosed or referenced herein that arise by alternative splicing of an mRNA corresponding to a DNA. A fragment of a polynucleotide includes a polynucleotide sequence that comprises a contiguous sequence of approximately at least about 6 nucleotides, in particular at least about 8 nucleotides, more particularly at least about 10-12 or 10 to 20 nucleotides, that correspond to (i.e. identical or complementary to), a region of the specified nucleotide sequence.

“Significantly different” levels of markers or a “significant difference” in marker levels in a patient sample compared to a control or standard (e.g. normal levels, levels from a different disease stage, or levels in other samples from a patient) may represent levels that are higher or lower than the standard error of the detection assay, preferably the levels are at least about 1.5, 2, 3, 4, 5, 6, 7, 8, 9 or 10 times higher or lower, respectively, than the control or standard.

“Microarray” and “array,” refer to nucleic acid or nucleotide arrays or protein or peptide arrays that can be used to detect Epithelial Cancer Markers associated with epithelial cancer, for instance to measure gene or protein expression. A variety of arrays are available commercially, such, for example, as the in situ synthesized oligonucleotide array GeneChip™ made by Affymetrix, Inc. or the spotted cDNA array, LifeArray™ made by Incyte Genomics Inc.

“Binding agent” refers to a substance such as a polypeptide, antibody, ribosome, or aptamer that specifically binds to an Ep-ICD polypeptide. A substance “specifically binds” to an Ep-ICD polypeptide if it reacts at a detectable level with the polypeptide, and does not react detectably with peptides containing unrelated sequences or sequences of different polypeptides. Binding properties may be assessed using an ELISA, which may be readily performed by those skilled in the art.

A binding agent may be a ribosome, with or without a peptide component, RNA or DNA molecule, or a polypeptide. A binding agent may be a polypeptide that comprises an Ep-ICD polypeptide sequence, a peptide variant thereof, or a non-peptide mimetic of such a sequence. By way of example, an Ep-ICD polypeptide sequence may be a peptide portion of the polypeptide that is capable of modulating a function mediated by the polypeptide.

An aptamer includes a DNA or RNA molecule that binds to nucleic acids and proteins. An aptamer that binds to an Epithelial Cancer Marker can be produced using conventional techniques, without undue experimentation. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

Antibodies include, but are not limited to, synthetic antibodies, polyclonal antibodies, monoclonal antibodies, recombinantly produced antibodies, intrabodies, multispecific antibodies (including bi-specific antibodies), human antibodies, humanized antibodies, chimeric antibodies (for example, antibodies which contain the binding specificity of murine antibodies, but in which the remaining portions are of human origin), single-chain Fvs (scFv) (including bi-specific scFvs), single chain antibodies, immunologically active fragments (e.g. Fab fragments, F(ab′) fragments, (Fab)₂ fragments), antibody light chains, disulfide-linked Fvs (sdFv), and anti-idiotypic (anti-Id) antibodies, and epitope-binding fragments of any of the above, or derivatives, such as enzyme conjugates or labelled derivatives. In particular, antibodies include immunoglobulin molecules and immunologically active portions of immunoglobulin molecules, i.e., molecules that contain an antigen binding site that immunospecifically bind to an Ep-ICD Polypeptide.

Antibodies including monoclonal and polyclonal antibodies, fragments and chimeras, may be prepared using methods well known to those skilled in the art. Isolated native or recombinant Ep-ICD polypeptides may be utilized to prepare antibodies. See, for example, Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. Antibodies specific for Ep-ICD polypeptides may also be obtained from scientific or commercial sources. In an embodiment of the invention, antibodies are reactive against Ep-ICD polypeptides if they bind with a Kₐ of greater than or equal to 10⁻⁷ M.

“Prognosis”, as used herein, refers to a prediction of the probable course and/or outcome of a disease. In some embodiments, a poor prognosis would predict that one or more of disease recurrence, death, and metastasis (in the case of cancer) will occur within about five years. In some embodiments, a favourable or good prognosis would predict that disease recurrence, death, and metastasis (in the case of cancer) will not occur within about five years.

General Methods

A variety of methods can be employed for the diagnostic and prognostic evaluation of an epithelial cancer and the identification of subjects with a predisposition to such conditions. Such methods may, for example, utilize Ep-ICD polynucleotides and fragments thereof, and binding agents (e.g. antibodies) directed against Ep-ICD polypeptides including peptide fragments. In particular, the polynucleotides and antibodies may be used, for example, for (1) the detection of the presence of polynucleotide mutations, or the detection of either over- or under-expression of mRNA, relative to a non-disorder state or the qualitative or quantitative detection of alternatively spliced forms of polynucleotide transcripts which may correlate with certain conditions or susceptibility toward such conditions; and (2) the detection of either an over- or an under-abundance of polypeptides relative to a non-disorder state or the presence of a modified (e.g., less than full length) polypeptide which correlates with a disorder state, or a progression toward a disorder state.

The methods described herein may be used to evaluate the probability of the presence of malignant cells, for example, in a group of cells freshly removed from a host. Such methods can be used to detect tumors, quantitate and monitor their growth, and help in the diagnosis and prognosis of disease. For example, higher levels of Ep-ICD are indicative of an epithelial cancer or metastatic epithelial cancer, in particular breast cancer, head and neck cancer or prostate cancer.

In an aspect, the invention contemplates a method for determining the aggressiveness or stage of epithelial cancer comprising producing a profile of levels of Ep-ICD polypeptides, and other markers associated with epithelial cancer, in cells from a patient, and comparing the profile with a reference to identify a profile for the test cells indicative of aggressiveness or stage of disease.

The methods of the invention require that the amount of Epithelial Cancer Markers quantitated in a sample from a subject being tested be compared to a predetermined standard or cut-off value. A standard may correspond to levels quantitated for another sample or an earlier sample from the subject, or levels quantitated for a control sample, in particular a sample from a subject with a lower grade cancer. Levels for control samples from healthy subjects or cancer subjects may be established by prospective and/or retrospective statistical studies. Healthy subjects who have no clinically evident disease or abnormalities may be selected for statistical studies. Diagnosis may be made by a finding of statistically different levels of Epithelial Cancer Markers compared to a control sample or previous levels quantitated for the same subject.

The invention also contemplates the methods described herein using multiple markers for epithelial cancer. Therefore, the invention contemplates a method for analyzing a biological sample for the presence of Epithelial Cancer Markers and other markers that are specific indicators of an epithelial cancer. The methods described herein may be modified by including reagents to detect the markers or polynucleotides encoding the markers. Other markers for breast cancer include, without limitation, BRCA1, BRCA2, urokinase plasminogen activator, plasminogen activator inhibitor, and CA27.29. Other markers for prostate cancer include, without limitation, prostate-specific antigen (PSA). Other markers for ovarian cancer include, without limitation, CA-125. The other markers may include EpCAM and EpEx.

Nucleic Acid Methods

As noted herein an epithelial cancer may be detected based on the level of Ep-ICD polynucleotides in a sample. Techniques for detecting nucleic acid molecules such as polymerase chain reaction (PCR) and hybridization assays are well known in the art.

Probes may be used in hybridization techniques to detect polynucleotides. The technique generally involves contacting and incubating nucleic acids obtained from a sample from a patient or other cellular source with a probe under conditions favorable for the specific annealing of the probes to complementary sequences in the nucleic acids (e.g. under stringent conditions as discussed herein). After incubation, the non-annealed nucleic acids are removed, and the presence of nucleic acids that have hybridized to the probe if any are detected.

Nucleotide probes for use in the detection of polynucleotide sequences in samples may be constructed using conventional methods known in the art. The probes may comprise DNA or DNA mimics corresponding to a portion of an organism's genome, or complementary RNA or RNA mimics. The nucleic acids can be modified at the base moiety, at the sugar moiety, or at the phosphate backbone. DNA can be obtained using standard methods such as polymerase chain reaction (PCR) amplification of genomic DNA or cloned sequences. Computer programs known in the art can be used to design primers with the required specificity and optimal amplification properties.

A nucleotide probe may be labeled with a detectable substance such as a radioactive label which provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleic acids to be detected and the amount of nucleic acids available for hybridization. Labeled probes may be hybridized to nucleic acids on solid supports such as nitrocellulose filters or nylon membranes as generally described in Sambrook et al., 1989, Molecular Cloning, A Laboratory Manual (2nd ed.). The nucleic acid probes may be used to detect Ep-ICD polynucleotides, preferably in human cells. The nucleotide probes may also be useful in the diagnosis of epithelial cancer, involving Ep-ICD polynucleotides in monitoring the progression of epithelial cancer, or monitoring a therapeutic treatment.

The detection of polynucleotides in a sample may involve the amplification of specific gene sequences using an amplification method such as PCR, followed by the analysis of the amplified molecules using techniques known to those skilled in the art. By way of example, oligonucleotide primers may be employed in a PCR based assay to amplify a portion of a polynucleotide and to amplify a portion of a polynucleotide derived from a sample, wherein the oligonucleotide primers are specific for (i.e. hybridize to) the polynucleotides. The amplified cDNA is then separated and detected using techniques well known in the art, such as gel electrophoresis.

In order to maximize hybridization under assay conditions, primers and probes employed in the methods of the invention generally have at least about 60%, preferably at least about 75% and more preferably at least about 90% identity to a portion of an Ep-ICD polynucleotide; that is, they are at least 10 nucleotides, and preferably at least 20 nucleotides in length. In an embodiment the primers and probes are at least about 10-40 nucleotides in length.

Hybridization and amplification reactions may also be conducted under stringent conditions as discussed herein.

Hybridization and amplification techniques described herein may be used to assay qualitative and quantitative aspects of polynucleotide expression. For example, RNA may be isolated from a cell type or tissue known to express Ep-ICD polynucleotides, and tested utilizing the hybridization (e.g. standard Northern analyses) or PCR techniques.

The primers and probes may be used in situ i.e., directly on tissue sections (fixed and/or frozen) of patient tissue obtained from biopsies or resections.

In an aspect of the invention, a method is provided employing reverse transcriptase-polymerase chain reaction (RT-PCR), in which PCR is applied in combination with reverse transcription. Generally, RNA is extracted from a sample tissue using standard techniques and is reverse transcribed to produce cDNA. The cDNA is used as a template for a polymerase chain reaction. The cDNA is hybridized to primer sets which are specifically designed against an Ep-ICD polynucleotide. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with suspected epithelial cancer, an individual who is not afflicted with epithelial cancer or has early stage disease or has aggressive or metastatic disease. The reaction may be performed on several dilutions of cDNA spanning at least two orders of magnitude. A statistically significant difference in expression in several dilutions of the subject sample as compared to the same dilutions of the non-cancerous sample or early-stage cancer sample may be considered positive for the presence of cancer.

Oligonucleotides or longer fragments derived from Ep-ICD polynucleotides may be used as targets in a microarray. The microarray can be used to monitor the expression levels of the polynucleotides and to identify genetic variants, mutations, and polymorphisms. The information from the microarray may be used to determine gene function, to understand the genetic basis of a disorder, to diagnose a disorder, and to develop and monitor the activities of therapeutic agents. Thus, the invention also includes an array comprising Ep-ICD polynucleotides, and optionally other epithelial cancer markers. The array can be used to assay expression of Ep-ICD polynucleotides in the array. The invention allows the quantitation of expression of the polynucleotides.

The invention provides microarrays comprising Ep-ICD polynucleotides. In one embodiment, the invention provides a microarray for distinguishing samples associated with epithelial cancer comprising a positionally-addressable array of polynucleotide probes bound to a support, the polynucleotide probes comprising sequences complementary and hybridizable to Ep-ICD polynucleotides.

In an embodiment, the array can be used to monitor the time course of expression of Ep-ICD polynucleotides in the array. This can occur in various biological contexts such as tumor progression. An array can also be useful for ascertaining differential expression patterns of Ep-ICD polynucleotides, and optionally other epithelial cancer markers in normal and abnormal cells. This may provide a battery of nucleic acids that could serve as molecular targets for diagnosis or therapeutic intervention.

The preparation, use, and analysis of microarrays are well known to those skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505; Heller, R. A. et al. (1997) Proc. Natl. Acad. Sci. 94:2150-2155; and Heller, M. J. et al. (1997) U.S. Pat. No. 5,605,662.)

Protein Methods

Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, NY, 1988). In general, the presence or absence of an epithelial cancer in a subject may be determined by (a) contacting a sample from the subject with a binding agent; (b) detecting in the sample a level of polypeptide that binds to the binding agent; and (c) comparing the level of polypeptide with a predetermined standard or cut-off value. In particular aspects of the invention, the binding agent is an antibody.

In an aspect, the invention provides a diagnostic method for monitoring or diagnosing epithelial cancer in a subject by quantitating Ep-ICD polypeptides in a biological sample from the subject comprising reacting the sample with antibodies specific for Ep-ICD polypeptides which are directly or indirectly labeled with detectable substances and detecting the detectable substances.

In an aspect of the invention, a method for detecting or diagnosing or prognosing an epithelial cancer is provided comprising or consisting essentially of:
(a) obtaining a sample suspected of containing Ep-ICD polypeptides;
(b) contacting said sample with antibodies that specifically bind Ep-ICD polypeptides under conditions effective to bind the antibodies and form complexes;
(c) measuring the amount of Ep-ICD polypeptides present in the sample by quantitating the amount of the complexes; and
(d) comparing the amount of Ep-ICD polypeptides present in the samples with the amount of Ep-ICD polypeptides in a control, wherein a change or significant difference in the amount of Ep-ICD polypeptides in the sample compared with the amount in the control is indicative of an epithelial cancer or risk of an epithelial cancer.

In an embodiment, the invention contemplates a method of prognosing breast cancer in a subject. In some embodiments, the method comprises detecting the presence or absence of nuclear Ep-ICD in a biological sample from the subject, the biological sample comprising breast epithelial cells, wherein the presence of nuclear Ep-ICD is associated with a poor prognosis.

In some embodiments, the method comprises detecting a level of nuclear Ep-ICD in a biological sample from the subject, comparing the level detected in the biological sample to a control; and prognosing breast cancer based on the comparison between the detected level of nuclear Ep-ICD and the control.

In some embodiments, the control is a reference value indicative of the level of nuclear Ep-ICD in cancerous breast epithelial cells. In some embodiments, the control is a reference value indicative of the level of nuclear Ep-ICD in non-cancerous breast epithelial cells.

In an embodiment, the invention contemplates a method for monitoring the progression of epithelial cancer in an individual, comprising:
(a) contacting antibodies which bind to Ep-ICD polypeptides with a sample from the individual so as to form complexes comprising the antibodies and Ep-ICD polypeptides in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of disease, disease stage, progression, aggressiveness and/or metastatic potential of the cancer in said individual.

The amount of complexes may also be compared to a value representative of the amount of the complexes from an individual not at risk of, or afflicted with epithelial cancer at a different stage.

Antibodies specifically reactive with Ep-ICD polypeptides or derivatives, such as enzyme conjugates or labeled derivatives, may be used to detect Ep-ICD polypeptides in various samples (e.g. biological materials, in particular tissue samples). They may be used as diagnostic or prognostic reagents and they may be used to detect abnormalities in the level of Ep-ICD polypeptides or abnormalities in the structure, and/or temporal, tissue, cellular, or subcellular location of Ep-ICD polypeptides. Antibodies may also be used to screen potentially therapeutic compounds in vitro to determine their effects on epithelial cancer involving Ep-ICD polypeptides. In vitro immunoassays may also be used to assess or monitor the efficacy of particular therapies.

Antibodies may be used in any immunoassay that relies on the binding interaction between antigenic determinants of Ep-ICD polypeptides and the antibodies. Immunoassay procedures for in vitro detection of antigens in samples are also well known in the art. [See for example, Paterson et al., Int. J. Can. 37:659 (1986) and Burchell et al., Int. J. Can. 34:763 (1984) for a general description of immunoassay procedures]. Qualitative and/or quantitative determinations of Ep-ICD polypeptides in a sample may be accomplished by competitive or non-competitive immunoassay procedures in either a direct or indirect format. Detection of Ep-ICD polypeptides using antibodies can, for example involve immunoassays which are run in either the forward, reverse or simultaneous modes. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. Alternatively, the binding of antibodies to Ep-ICD polypeptides can be detected directly using, for example, a surface plasmon resonance (SPR) procedure such as, for example, Biacore®, microcalorimetry or nano-cantilivers. These terms are well understood by those skilled in the art, and they will know, or can readily discern, other immunoassay formats without undue experimentation.

Antibodies specific for Ep-ICD polypeptides may be labelled with a detectable substance and localised in biological samples based upon the presence of the detectable substance. Examples of detectable substances include, but are not limited to, the following: radioisotopes (e.g., ³H, ¹⁴C, ³⁵S, ¹²I, ¹³¹I), fluorescent labels, (e.g., FITC, rhodamine, lanthanide phosphors), luminescent labels such as luminol; and enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), and predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

One of the ways an antibody can be detectably labelled is to link it directly to an enzyme. The enzyme when later exposed to its substrate will produce a product that can be detected. Examples of detectable substances that are enzymes are horseradish peroxidase, beta-galactosidase, luciferase, alkaline phosphatase, acetylcholinesterase, malate dehydrogenase, ribonuclease, urease, catalase, glucose-6-phosphate, staphylococcal nuclease, delta-5-steroid isomerase, yeast alcohol dehydrogenase, alpha-glycerophosphate, triose phosphate isomerase, asparaginase, glucose oxidase, and acetylcholine esterase.

For increased sensitivity in an immunoassay system a fluorescence-emitting metal atom such as Eu (europium) and other lanthanides can be used. These can be attached to the desired molecule by means of metal-chelating groups such as DTPA or EDTA.

A bioluminescent compound may also be used as a detectable substance. Examples of bioluminescent detectable substances are luciferin, luciferase and aequorin.

Indirect methods may also be employed in which the primary antigen-antibody reaction is amplified by the introduction of a second antibody, having specificity for the antibody reactive against an Ep-ICD polypeptide. By way of example, if the antibody having specificity against an Ep-ICD polypeptide is a rabbit IgG antibody, the second antibody may be goat anti-rabbit IgG, Fc fragment specific antibody labeled with a detectable substance as described herein.

Methods for conjugating or labelling the antibodies discussed above may be readily accomplished by one of ordinary skill in the art.

Cytochemical techniques known in the art for localizing antigens using light and electron microscopy may be used to detect Ep-ICD polypeptides. Generally, an antibody may be labeled with a detectable substance and an Ep-ICD polypeptide may be localized in tissues and cells based upon the presence of the detectable substance.

In the context of the methods of the invention, the sample, binding agents (e.g. antibodies), or Ep-ICD polypeptides may be immobilized on a carrier or support, such as, for example, agarose, cellulose, nitrocellulose, dextran, Sephadex, Sepharose, liposomes, carboxymethyl cellulose, polyacrylamides, polystyrene, filter paper, ion-exchange resin, plastic film, nylon or silk. The support material may have any possible configuration including spherical cylindrical or flat. Thus, the carrier may be in the shape of, for example, a tube, test plate, well, beads, disc, sphere, etc. The immobilized material may be prepared by reacting the material with a suitable insoluble carrier using known chemical or physical methods, for example, cyanogen bromide coupling. Binding agents (e.g. antibodies) may be indirectly immobilized using second binding agents specific for the first binding agent. For example, mouse antibodies specific for Ep-ICD polypeptides may be immobilized using sheep anti-mouse IgG Fc fragment specific antibody coated on the carrier or support.

Where a radioactive label is used as a detectable substance, an Ep-ICD polypeptide may be localized by radioautography. The results of radioautography may be quantitated by determining the density of particles in the radioautographs by various optical methods, or by counting the grains.

Time-resolved fluorometry may be used to detect a fluorescent signal, label, or detectable substance. For example, the method described in Christopoulos T K and Diamandis EP Anal. Chem., 1992:64:342-346 may be used with a conventional time-resolved fluorometer.

According to an embodiment of the invention, an immunoassay for detecting a Ep-ICD polypeptide in a biological sample comprises contacting an amount of a binding agent that specifically binds to a Ep-ICD polypeptide in the sample under conditions that allow the formation of complexes comprising the binding agent and Ep-ICD polypeptide and determining the presence or amount of the complexes as a measure of the amount of the Ep-ICD polypeptide contained in the sample.

In accordance with an embodiment of the invention, a method is provided wherein Ep-ICD polypeptides antibodies are directly or indirectly labelled with enzymes, substrates for the enzymes are added wherein the substrates are selected so that the substrates, or a reaction product of an enzyme and substrate, form fluorescent complexes with lanthanide metals, preferably europium and terbium. A lanthanide metal(s) is added and Ep-ICD polypeptides are quantitated in the sample by measuring fluorescence of the fluorescent complexes. Enzymes are selected based on the ability of a substrate of the enzyme, or a reaction product of the enzyme and substrate, to complex with lanthanide metals.

Examples of enzymes and substrates for enzymes that provide such fluorescent complexes are described in U.S. Pat. No. 5,312,922 to Diamandis. By way of example, when the antibody is directly or indirectly labelled with alkaline phosphatase, the substrate employed in the method may be 4-methylumbelliferyl phosphate, 5-fluorosalicyl phosphate, or difiunisal phosphate. The fluorescence intensity of the complexes is typically measured using a time-resolved fluorometer.

Antibodies specific for Ep-ICD polypeptides may also be indirectly labelled with enzymes. For example, an antibody may be conjugated to one partner of a ligand binding pair, and the enzyme may be coupled to the other partner of the ligand binding pair. Representative examples include avidin-biotin, and riboflavin-riboflavin binding protein.

Aspects of the methods of the invention involve (a) reacting a biological sample from a subject with antibodies specific for Ep-ICD polypeptides wherein the antibodies are directly or indirectly labelled with enzymes; (b) adding substrates for the enzymes wherein the substrates are selected so that the substrates, or reaction products of the enzymes and substrates form fluorescent complexes; (c) quantitating Ep-ICD polypeptides in the sample by measuring fluorescence of the fluorescent complexes; and (d) comparing the quantitated levels to levels obtained for other samples from the subject, or control subjects. In an embodiment, the Ep-ICD polypeptide is nuclear Ep-ICD and the quantitated level is compared to levels quantitated for normal subjects or subjects with an early stage of disease wherein an increase in the level of nuclear Ep-ICD compared with the control subjects is indicative of an epithelial cancer and/or poor prognosis or survival.

In some embodiments, methods of the invention further comprise detecting EpEx in the biological sample from the subject. In some embodiments, the method for prognosing breast cancer in a subject further comprises detecting a level of EpEx in a sample from the subject and comparing the detected level of EpEx with a control. In some embodiments, the method comprises detecting the presence or absence of membranous EpEx in a sample from the subject.

In some embodiments, the method comprises detecting loss of membranous EpEx in a sample from the subject.

A particular embodiment of the invention comprises the following steps:
(a) incubating a biological sample with a first antibody specific for Ep-ICD polypeptides which is directly or indirectly labeled with a detectable substance, and a second antibody specific for Ep-ICD polypeptides which is immobilized;
(b) separating the first antibody from the second antibody to provide a first antibody phase and a second antibody phase;
(c) detecting the detectable substance in the first or second antibody phase thereby quantitating Ep-ICD polypeptides in the biological sample; and
(d) comparing the quantitated Ep-ICD polypeptides with levels for a predetermined standard.

The standard may correspond to levels quantitated for samples from control subjects with no disease or early stage disease or from other samples of the subject. Increased levels of Ep-ICD as compared to the standard may be indicative of an epithelial cancer or risk of an epithelial cancer.

In some embodiments, the invention further comprises the following steps:
(e) incubating a biological sample with a first antibody specific for EpEx polypeptides which is directly or indirectly labeled with a detectable substance, and a second antibody specific for EpEx polypeptides which is immobilized;
(f) separating the first antibody from the second antibody to provide a first antibody phase and a second antibody phase;
(g) detecting the detectable substance in the first or second antibody phase thereby quantitating EpEx polypeptides in the biological sample.

In some embodiments, the invention further comprises the following step:
(h) comparing the quantitated EpEx polypeptides with levels for a predetermined standard.

In some embodiments, the invention comprises a step of detecting membranous EpEx and/or loss of membranous EpEx in the biological sample.

In accordance with an embodiment, the present invention provides means for determining Ep-ICD polypeptides in a sample by measuring Ep-ICD polypeptides by immunoassay. It will be evident to a skilled artisan that a variety of competitive or non-competitive immunoassay methods can be used to measure Ep-ICD polypeptides in samples, in particular fluid samples such as serum. Competitive methods typically employ immobilized or immobilizable antibodies to Ep-ICD polypeptides and labeled forms of Ep-ICD polypeptides. Sample Ep-ICD polypeptides and labeled Ep-ICD polypeptides compete for binding to antibodies specific for Ep-ICD polypeptides. After separation of the resulting labeled Ep-ICD polypeptides that have become bound to antibody (bound fraction) from that which has remained unbound (unbound fraction), the amount of the label in either bound or unbound fraction is measured and may be correlated with the amount of Ep-ICD polypeptides in the test sample in any conventional manner, e.g., by comparison to a standard curve.

In another aspect, a non-competitive method is used for the determination of Ep-ICD polypeptides with the most common method being the “sandwich” method. In this assay, two antibodies specific for an Ep-ICD polypeptide are employed. One of the antibodies is directly or indirectly labeled (the “detection antibody”), and the other is immobilized or immobilizable (the “capture antibody”). The capture and detection antibodies can be contacted simultaneously or sequentially with the test sample. Sequential methods can be accomplished by incubating the capture antibody with the sample, and adding the detection antibody at a predetermined time thereafter or the detection antibody can be incubated with the sample first and then the capture antibody added. After the necessary incubation(s) have occurred, to complete the assay, the capture antibody may be separated from the liquid test mixture, and the label may be measured in at least a portion of the separated capture antibody phase or the remainder of the liquid test mixture. Generally it is measured in the capture antibody phase since it comprises Ep-ICD polypeptide “sandwiched” between the capture and detection antibodies. In another embodiment, the label may be measured without separating the capture antibody and liquid test mixture.

In particular sandwich immunoassays of the invention mouse polyclonal/monoclonal antibodies specific for Ep-ICD polypeptides and rabbit polyclonal/monoclonal antibodies specific for Ep-ICD polypeptides are utilized.

In a typical two-site immunometric assay for Ep-ICD polypeptides one or both of the capture and detection antibodies are polyclonal antibodies or one or both of the capture and detection antibodies are monoclonal antibodies (i.e. polyclonal/polyclonal, monoclonal/monoclonal, or monoclonal/polyclonal). The label used in the detection antibody can be selected from any of those known conventionally in the art. The label may be an enzyme or a chemiluminescent moiety, but it can also be a radioactive isotope, a fluorophor, a detectable ligand (e.g., detectable by a secondary binding by a labeled binding partner for the ligand), and the like. In an aspect, the antibody is labelled with an enzyme which is detected by adding a substrate that is selected so that a reaction product of the enzyme and substrate forms fluorescent complexes. The capture antibody may be selected so that it provides a means for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody may comprise an antibody covalently or noncovalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, the capture antibody may be immobilized using a species specific antibody for the capture antibody that is bound to the solid phase.

Screening Methods

The invention also contemplates methods for evaluating test agents or compounds for their potential efficacy in treating an epithelial cancer. Test agents and compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], polynucleotides (e.g. antisense, siRNA), and small organic or inorganic molecules. The agents or compounds may be endogenous physiological compounds or natural or synthetic compounds.

The invention provides a method for assessing the potential efficacy of a test agent in treating epithelial cancer comprising comparing:
(a) levels of one or more Epithelial Cancer Markers, and optionally other markers in a first sample obtained from a patient and exposed to the test agent; and
(b) levels of one or more Epithelial Cancer Markers, and optionally other markers, in a second sample obtained from the patient, wherein the sample is not exposed to the test agent, wherein a significant difference in the levels of expression of one or more Epithelial Cancer Markers, and optionally the other markers, in the first sample, relative to the second sample, is an indication that the test agent is potentially efficacious for treating epithelial cancer in the patient.

The first and second samples may be portions of a single sample obtained from a patient or portions of pooled samples obtained from a patient(s).

In an aspect, the invention provides a method of selecting an agent for treating epithelial cancer in a patient comprising:
(a) obtaining a sample from the patient;
(b) separately maintaining aliquots of the sample in the presence of a plurality of test agents;
(c) comparing one or more Epithelial Cancer Markers, and optionally other markers, in each of the aliquots; and
(d) selecting one of the test agents which alters the levels of one or more Epithelial Cancer Markers, and optionally other markers in the aliquot containing that test agent, relative to other test agents.

Kits

The invention contemplates kits for carrying out the methods of the invention to detect an epithelial cancer. Such kits typically comprise two or more components required for performing a diagnostic or prognostic assay. Components include but are not limited to one or more of compounds, reagents, containers, equipment and instructions. Accordingly, the methods described herein may be performed by utilizing pre-packaged diagnostic kits comprising at least agents (e.g. antibodies, probes, primers, etc) described herein, which may be conveniently used, e.g., in clinical settings to diagnose patients afflicted with epithelial cancer, or exhibiting a predisposition to developing epithelial cancer and in particular to diagnose an epithelial cancer or risk of an epithelial cancer, or to prognose breast cancer patients.

The invention contemplates a container with a kit comprising a binding agent(s) as described herein for determining an epithelial cancer, including breast cancer. By way of example, the kit may contain antibodies specific for Ep-ICD polypeptides, antibodies against the antibodies labelled with an enzyme(s), and a substrate for the enzyme(s). The kit may also contain microtiter plate wells, standards, assay diluent, wash buffer, adhesive plate covers, and/or instructions for carrying out a method of the invention using the kit.

In an aspect, the invention provides a test kit for diagnosing an epithelial cancer in a subject which comprises an antibody that binds to Ep-ICD polypeptides and/or polynucleotides that hybridize to or amplify Ep-ICD polynucleotides. In another aspect the invention relates to use of an antibody that binds to a Ep-ICD polypeptide and/or a polynucleotide that hybridizes to or amplifies a Ep-ICD polynucleotide, in the manufacture of a composition for detecting an epithelial cancer including detecting the aggressiveness or metastatic potential of a epithelial cancer.

In a further aspect of the invention, the kit includes antibodies or antibody fragments which bind specifically to epitopes of Ep-ICD polypeptides and means for detecting binding of the antibodies to their epitopes associated with epithelial cancer cells, either as concentrates (including lyophilized compositions), which may be further diluted prior to testing. In particular, the invention provides a kit for diagnosing an epithelial cancer comprising a known amount of a first binding agent that specifically binds to Ep-ICD polypeptides wherein the first binding agent comprises a detectable substance, or it binds directly or indirectly to a detectable substance.

A kit may be designed to detect the levels of Ep-ICD polynucleotides in a sample. Such kits generally comprise oligonucleotide probes or primers, as described herein, which hybridize to or amplify Ep-ICD polynucleotides. Oligonucleotides may be used, for example, within PCR or hybridization procedures. Test kits useful for detecting target Ep-ICD polynucleotides are also provided which comprise a container containing Ep-ICD polynucleotide, and fragments or complements thereof. A kit can comprise one or more primers.

In a further aspect of the invention, one or more of the kits described herein can be used for prognosis of breast cancer. In some embodiments, a kit for prognosis of breast cancer includes one or more of compounds, reagents, containers, equipment and instructions. Accordingly, the methods described herein may be performed by utilizing pre-packaged prognostic kits comprising one or more of agents (e.g. antibodies, probes etc.), standards, stains, fixatives and instructions. In some embodiments, the instructions comprise one or more reference values for use as controls.

In some embodiments, the kit comprises one or more agents for detecting nuclear Ep-ICD. In some embodiments, the kit comprises one or more agents for detecting membranous EpEx.

The kits of the invention can further comprise containers with tools useful for collecting test samples (e.g. serum) including lancets and absorbent paper or cloth for collecting and stabilizing blood.

Computer Systems

Analytic methods contemplated herein can be implemented by use of computer systems and methods described below and known in the art. Thus, the invention provides computer readable media comprising one or more Epithelial Cancer Markers. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on one or more markers disclosed herein.

A variety of data processor programs and formats can be used to store information on one or more Epithelial Cancer Markers. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of data processor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining whether a patient has epithelial cancer, or a pre-disposition to such condition, comprising determining the presence or absence of one or more Epithelial Cancer Markers, and based on the presence or absence of the markers, determining the condition or a pre-disposition to the condition, optionally recommending a procedure or treatment.

The invention also provides in an electronic system and/or in a network, a method for determining whether a subject has a condition disclosed herein, or a pre-disposition to a condition disclosed herein, comprising determining the presence or absence of one or more markers, and based on the presence or absence of the markers, determining whether the subject has the condition or a pre-disposition to the condition, and optionally recommending a procedure or treatment.

The invention further provides in a network, a method for determining whether a subject has a condition disclosed herein or a pre-disposition to a condition disclosed herein comprising: (a) receiving phenotypic information on the subject and information on one or more markers disclosed herein associated with samples from the subject; (b) acquiring information from the network corresponding to the markers; and (c) based on the phenotypic information and information on the markers, determining whether the subject has the condition or a pre-disposition to the condition, and (d) optionally recommending a procedure or treatment.

The invention still further provides a system for identifying selected records that identify a diseased cell or tissue. A system of the invention generally comprises a digital computer; a database server coupled to the computer; a database coupled to the database server having data stored therein, the data comprising records of data comprising one or more markers disclosed herein, and a code mechanism for applying queries based upon a desired selection criteria to the data file in the database to produce reports of records which match the desired selection criteria.

The invention contemplates a business method for determining whether a subject has a condition disclosed herein or a pre-disposition to a condition disclosed herein comprising: (a) receiving phenotypic information on the subject and information on one or more markers disclosed herein associated with samples from the subject; (b) acquiring information from a network corresponding to the markers; and (c) based on the phenotypic information, information on the markers and acquired information, determining whether the subject has the condition or a pre-disposition to the condition, and optionally recommending a procedure or treatment.

In an aspect of the invention, the computer systems, components, and methods described herein are used to monitor a condition (i.e. epithelial cancer) or determine the stage of a condition.

Therapeutic Applications

The invention contemplates therapeutic applications associated with the Epithelial Cancer Markers disclosed herein. Epithelial Cancer Markers may be a target for therapy. For example, Ep-ICD can be a target for treatment of epithelial cancers. Therapeutic methods include immunotherapeutic methods including the use of antibody therapy. In one aspect, the invention provides one or more antibodies that may be used to treat or prevent epithelial cancer. In another aspect, the invention provides a method of preventing, inhibiting or reducing epithelial cancer comprising administering to a patient an antibody which binds to an Ep-ICD polypeptide in an amount effective to prevent, inhibit, or reduce the condition or the onset of the condition.

An antibody which binds to an Ep-ICD polypeptide may be in combination with a label, drug or cytotoxic agent, a target-binding region of a receptor, an adhesion molecule, a ligand, an enzyme, a cytokine, or a chemokine. In aspects of the invention, the antibody may be conjugated to cytotoxic agents (e.g., chemotherapeutic agents) or toxins or active fragments thereof. Examples of toxins and corresponding fragments thereof include diptheria A chain, exotoxin A chain, ricin A chain, abrin A chain, curcin, crotin, phenomycin, enomycin and the like. A cytotoxic agent may be a radiochemical prepared by conjugating radioisotopes to antibodies, or binding of a radionuclide to a chelating agent that has been covalently attached to the antibody. An antibody may also be conjugated to one or more small molecule toxins, such as a calicheamicin, a maytansine, a trichothene, and CC1065 (see U.S. Pat. No. 5,208,020).

The methods of the invention contemplate the administration of single antibodies as well as combinations, or “cocktails”, of different individual antibodies such as those recognizing different epitopes of other markers. Such cocktails may have certain advantages inasmuch as they contain antibodies that bind to different epitopes of Ep-ICD polypeptides and/or exploit different effector mechanisms. Such antibodies in combination may exhibit synergistic therapeutic effects. In addition, the administration of one or more marker specific antibodies may be combined with other therapeutic agents. The specific antibodies may be administered in their “naked” or unconjugated form, or may have therapeutic agents conjugated to them.

In an aspect, the invention contemplates a method of treating an epithelial cancer in a subject, comprising delivering to the subject in need thereof, an antibody specific for nuclear Ep-ICD. In an aspect of the invention, the antibody is conjugated to a cytotoxic agent or toxin (see above). The antibody may be a therapeutic antibody disclosed for example in U.S. Pat. No. 7,557,190 and U.S. Pat. No. 7,459,538, US Published Application Nos. 20050163785 and 20070122406, and 20070196366 and McDonald et al. (Drug Design, Development and Therapy 2008; 2:105-114). In a particular embodiment, the antibody is an antibody conjugated to a toxin, more particularly VB4-845 immunotoxin (Viventia Biotechnologies Inc., Ontario, Canada).

More particularly, and according to one aspect of the invention, there is provided a method of treating a subject having an epithelial cancer wherein an antibody specific for Ep-ICD is administered in a therapeutically effective amount. In a further aspect, the antibody is provided in a pharmaceutically acceptable form.

In an aspect, the invention provides a pharmaceutical composition for the treatment of an epithelial cancer characterized in that the composition comprises an antibody specific for Ep-ICD together with a pharmaceutically acceptable carrier, excipient or vehicle.

Antibodies used in the methods of the invention may be formulated into pharmaceutical compositions comprising a carrier suitable for the desired delivery method. Suitable carriers include any material which when combined with the antibodies retains the function of the antibody and is non-reactive with the subject's immune systems. Examples include any of a number of standard pharmaceutical carriers such as sterile phosphate buffered saline solutions, bacteriostatic water, and the like (see, generally, Remington: The Science and Practice of Pharmacy 21^{st} Edition. 2005, University of the Sciences in Philadelphia (Editor), Mack Publishing Company).

One or more marker specific antibody formulations may be administered via any route capable of delivering the antibodies to the site or injury. Routes of administration include, but are not limited to, intravenous, intraperitoneal, intramuscular, intradermal, and the like. Antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration at an effective dose. Dosages will depend upon various factors generally appreciated by those of skill in the art, including the etiology of the condition, stage of the condition, the binding affinity and half life of the antibodies used, the degree of marker expression in the patient, the desired steady-state antibody concentration level, frequency of treatment, and the influence of any therapeutic agents used in combination with a treatment method of the invention. A determining factor in defining the appropriate dose is the amount of a particular antibody necessary to be therapeutically effective in a particular context. Repeated administrations may be required to achieve a desired effect. Direct administration of one or more marker antibodies is also possible and may have advantages in certain situations.

Patients may be evaluated for Epithelial Cancer Markers in order to assist in the determination of the most effective dosing regimen and related factors. The assay methods described herein, or similar assays, may be used for quantitating marker levels in patients prior to treatment. Such assays may also be used for monitoring throughout therapy, and may be useful to gauge therapeutic success in combination with evaluating other parameters such as levels of markers.

Ep-ICD polynucleotides disclosed herein can be turned off by transfecting a cell or tissue with vectors that express high levels of the polynucleotides. Such constructs can inundate cells with untranslatable sense or antisense sequences. Even in the absence of integration into the DNA, such vectors may continue to transcribe RNA molecules until all copies are disabled by endogenous nucleases. Vectors derived from retroviruses, adenovirus, herpes or vaccinia viruses, or from various bacterial plasmids, may be used to deliver polynucleotides to a targeted organ, tissue, or cell population. Methods well known to those skilled in the art may be used to construct recombinant vectors that will express polynucleotides such as antisense. (See, for example, the techniques described in Sambrook et al (supra) and Ausubel et al (supra).)

Methods for introducing vectors into cells or tissues include those methods discussed herein and which are suitable for in vivo, in vitro and ex vivo therapy. For example, delivery by transfection or by liposome are well known in the art.

Modifications of gene expression can be obtained by designing antisense molecules, DNA, RNA or PNA, to the regulatory regions of a polynucleotide, i.e., the promoters, enhancers, and introns. Preferably, oligonucleotides are derived from the transcription initiation site, e.g. between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA are reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of a polynucleotide marker.

Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

The invention provides a method of preventing, inhibiting, or reducing epithelial cancer in a patient comprising:
(a) obtaining a tumor sample from the patient;
(b) separately maintaining aliquots of the sample in the presence of a plurality of test agents;
(c) comparing levels of Epithelial Cancer Markers, and optionally one or more other markers of the epithelial cancer, in each aliquot;
(d) administering to the patient at least one test agent which alters the levels of Epithelial Cancer Markers, and optionally other markers of the epithelial cancer, in the aliquot containing that test agent, relative to the other test agents.

An active therapeutic substance described herein may be administered in a convenient manner by any standard route of administration, including without limitation, by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance. Solutions of an active substance as a free base or pharmaceutically acceptable salt can be prepared in an appropriate solvent with a suitable surfactant. Dispersions may be prepared in glycerol, liquid polyethylene glycols, and mixtures thereof, or in oils.

A composition described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington: The Science and Practice of Pharmacy (21^{st} Edition. 2005, University of the Sciences in Philadelphia (Editor), Mack Publishing Company), and in The United States Pharmacopeia: The National Formulary (USP 24 NF19) published in 1999. On this basis, the compositions include, albeit not exclusively, solutions of the active substances in association with one or more pharmaceutically acceptable vehicles or diluents, and contained in buffered solutions with a suitable pH and iso-osmotic with the physiological fluids.

A composition is indicated as a therapeutic agent either alone or in conjunction with other therapeutic agents or other forms of treatment. The compositions of the invention may be administered concurrently, separately, or sequentially with other therapeutic agents or therapies.

The therapeutic activity of compositions and agents/compounds identified using a method of the invention and may be evaluated in vivo using a suitable animal model.

### EXAMPLES

The following non-limiting examples are illustrative of the present invention:

### Example 1

### Immunohistochemical Analysis of Epithelial Cancer Markers

The following materials and methods were employed in the study described in this example:

Antibodies

Anti-human-EpCAM mouse monoclonal antibody MOC-31 (AbD Serotec, Oxford, UK, Raleigh, N.C.) recognizes an extracellular component (EpEx-EGF1 domain-aa 27-59) in the amino-terminal region of EpCAM [Myklebust et al, Cancer Res. 1993 Aug. 15; 53(16):3784-8.]. Anti-human rabbit monoclonal antibody, α-Ep-ICD antibody 1144 (Epitomics, Burlingame, Calif.) recognizes the cytoplasmic domain of human EpCAM. β-catenin antibody was raised against aa 571-781 of β-catenin (Cat. #610154, B D Sciences, San Jose, Calif.).

Immunohistochemistry for EpEx and ED-ICD Expression in Epithelial Cancers

Serial epithelial cancer tissue sections (4 μm thickness) were deparaffinized, hydrated in xylene and graded alcohol series. The slides were treated with 0.3% H₂O₂ at room temperature for 30 minutes to block the endogenous peroxidase activity. After blocking the non-specific binding with normal horse or goat serum, the sections were incubated with anti human antibodies -EpEx mouse monoclonal antibody MOC-31 (dilution 1:200), or α-Ep-ICD rabbit monoclonal antibody 1144 (dilution 1:200), or mouse monoclonal β-catenin antibody (dilution 1:200) for 30 minutes and biotinylated secondary antibody (horse anti-mouse or goat antirabbit) for 30 minutes. The sections were finally incubated with VECTASTAIN Elite ABC Reagent (Vector labs, Burlingame, Calif.) and diaminobenzedine was used as the chromogen.

Evaluation of Immunohistochemical Staining

Immunopositive staining was evaluated in five areas of the tissue sections as described [Ralhan et al., 2008, J Proteome Res. 2009 January; 8(1):300-9]. Sections were scored as positive if epithelial cells showed immunopositivity in the plasma membrane, cytoplasm, and/or nucleus when observed by two evaluators who were blinded to the clinical outcome. These sections were scored as follows: 0, <10% cells; 1, 10-30% cells; 2, 30-50% cells; 3, 50-70% cells; and 4, >70% cells showed immunoreactivity. Sections were also scored semi-quantitatively on the basis of intensity as follows: 0, none; 1, mild; 2, moderate; and 3, intense. Finally, a total score (ranging from 0 to 7) was obtained by adding the scores of percentage positivity and intensity for each of the epithelial cancer and normal epithelial tissue sections. The immunohistochemical data were subjected to statistical analysis.

Statistical Analysis

The immunohistochemical data were subjected to statistical analysis using SPSS 10.0 software (Chicago). Box plots were used to determine the distribution of total score of membranous EpEx, nuclear Ep-ICD and nuclear or cytoplasmic β-catenin expression in normal tissues and tumors. A cut-off= or >2 was defined as positive criterion for nuclear β-catenin immunopositivity for statistical examination. For membranous β-catenin, score of 6 was defined as loss of expression. The correlation between expression of EpEx, Ep-ICD and/or β-catenin staining with overall patient survival was evaluated using life tables constructed from survival data with Kaplan-Meier plots.

Immunofluorescence Analysis

The immunofluorescence analysis of Ep-ICD and EpEx localization in human breast, colon and prostate carcinomas was performed fluorescent secondary antibodies. Human breast carcinoma, colon carcinoma and prostate carcinoma paraffin sections were incubated with either α-Ep-ICD rabbit monoclonal antibody 1144 (dilution 1:100) or mouse monoclonal antibody MOC-31 (dilution 1:100). For Ep-ICD, the secondary antibody used was a tetramethyl rhodamine isothiocyanate (TRITC)-labeled goat anti-rabbit antibody (Sigma-Aldrich, dilution 1:200). For EpEx, the secondary antibody used was a fluorescein isothiocyanate (FITC)-labeled goat anti-mouse antibody (Sigma-Aldrich, St. Louis, Mo., 1:200 dilution). Nuclei were counterstained with DAPI (blue). Localization of EpEx (green) and Ep-ICD (red) were assessed with Olympus Upright Flourescence Microscope (BX61) and images were captured by using Volocity software (PerkinElmer Waltham, Mass.)

Results

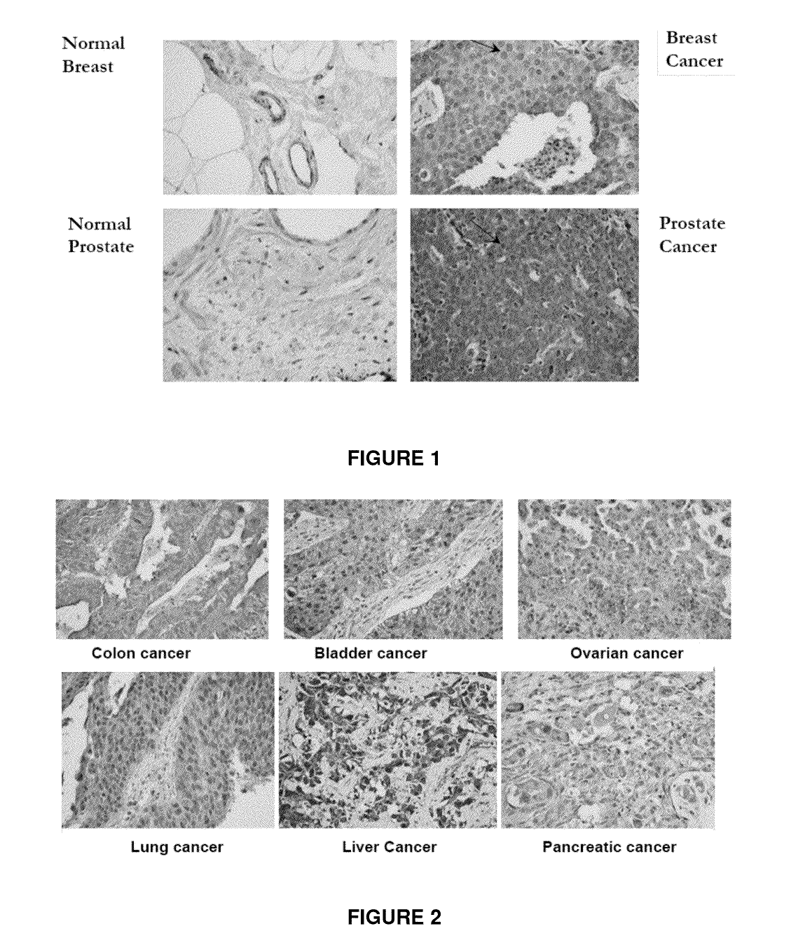

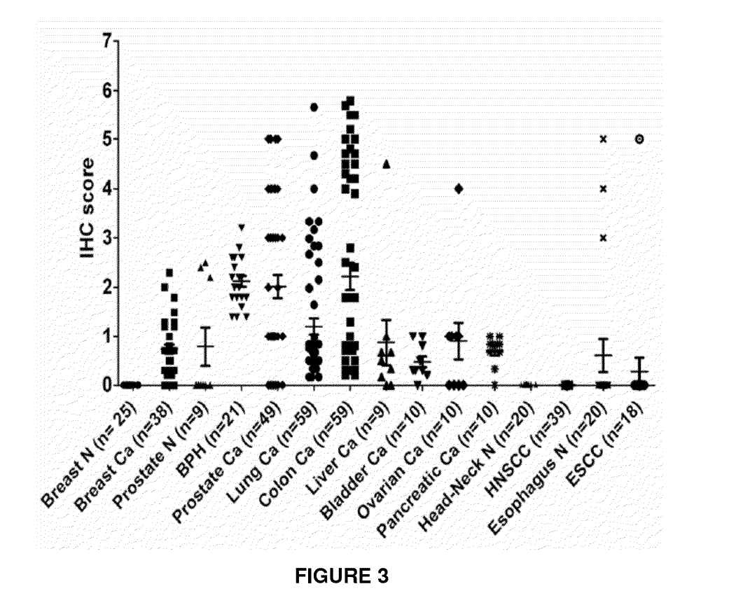

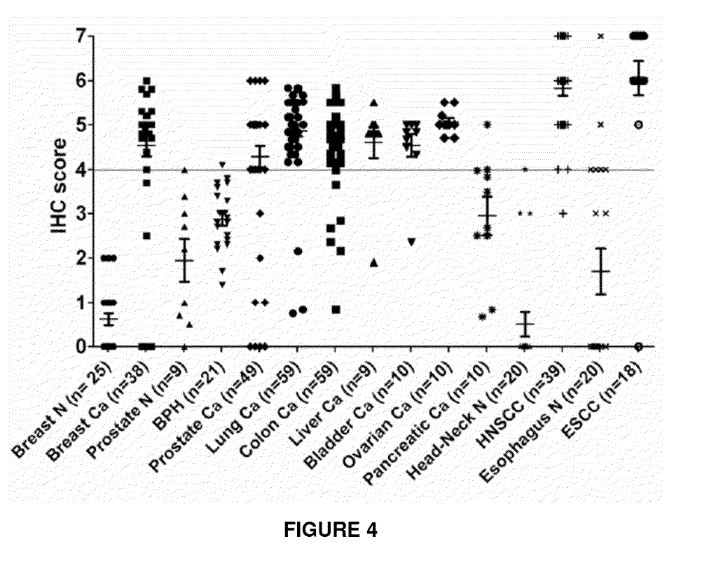

Immunohistochemical staining of paraffin tissue sections from a range of patients was conducted with a full-length monoclonal antibody to EpCAM (Ep-Ex) and a monoclonal antibody to the intracellular domain of EpCAM (Ep-ICD). Scoring analyses was performed by two independent observers for degrees of subcellular localization to the cell membrane, cytoplasm or nucleus. Nuclear Ep-ICD was detected in tissues from patients with breast, prostate, colon and rectum, lung, pancreatic, urinary bladder, ovarian, liver and head and neck cancers. The results are shown in FIGS. 1 to 11 and the following Tables 1-6. FIG. 1 shows nuclear Ep-ICD expression in representative breast and prostate tissue sections. Nuclear Ep-ICD was also observed in tissues from patients with cancers of colon and rectum, urinary bladder, ovarian, lung, liver and pancreas (FIG. 2), suggesting that nuclear Ep-ICD expression can be detected in several epithelial cancers. The distribution of Ep-ICD in plasma membrane of epithelial cells in normal and malignant breast tissues, normal and malignant prostate tissues, and in cancers of lung, colon and rectum, liver, urinary bladder, ovary and pancreas analysed are shown in the bar diagram in FIG. 3. The cytoplasmic distribution of Ep-ICD in epithelial cells in normal and malignant breast tissues, normal and malignant prostate tissues, and in cancers of lung, colon and rectum, liver, urinary bladder, ovary and pancreas analysed are shown in the bar diagram in FIG. 4.

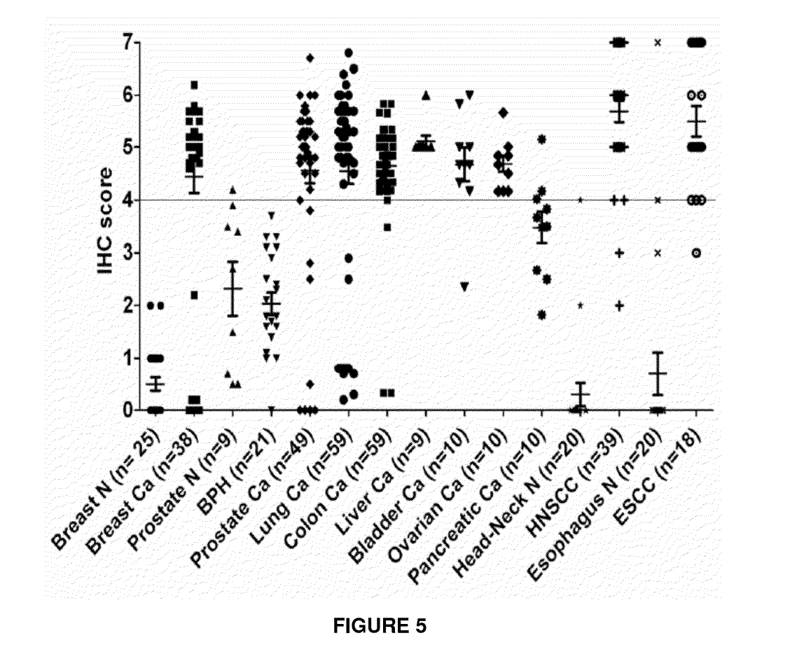

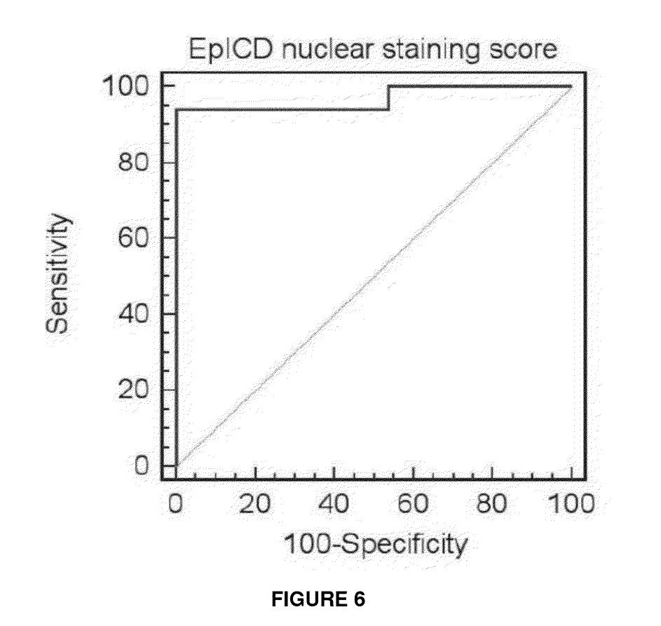

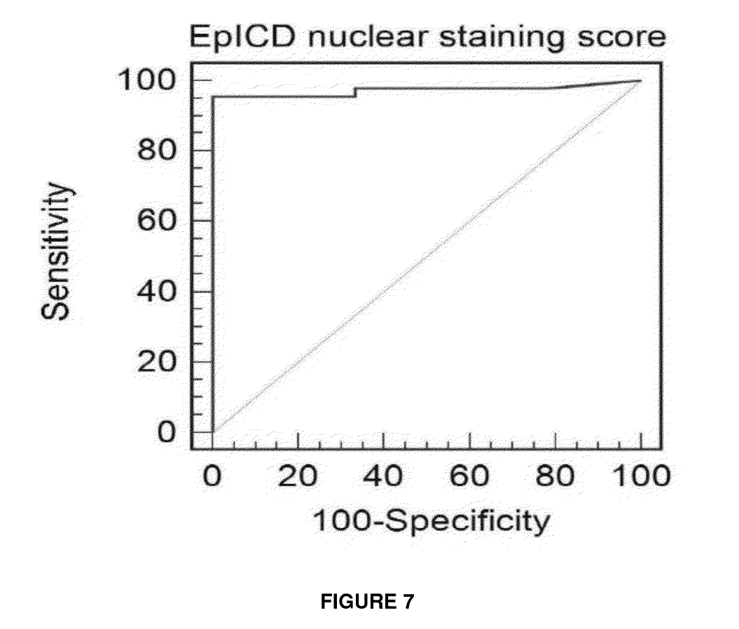

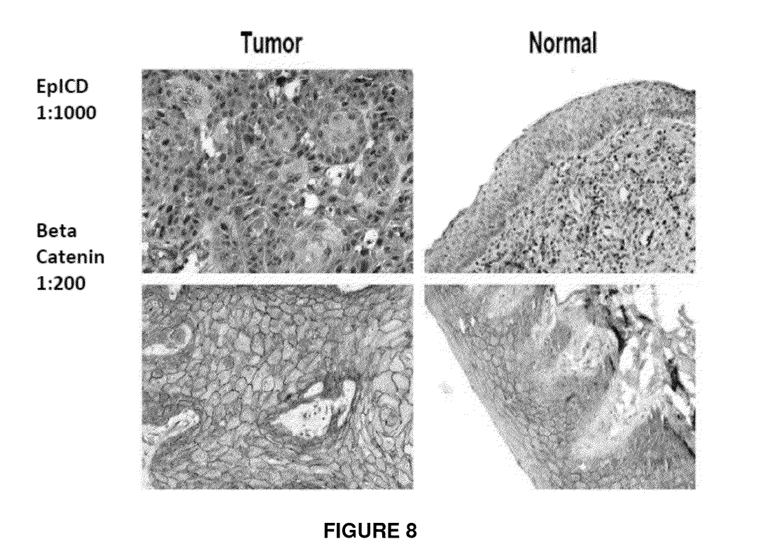

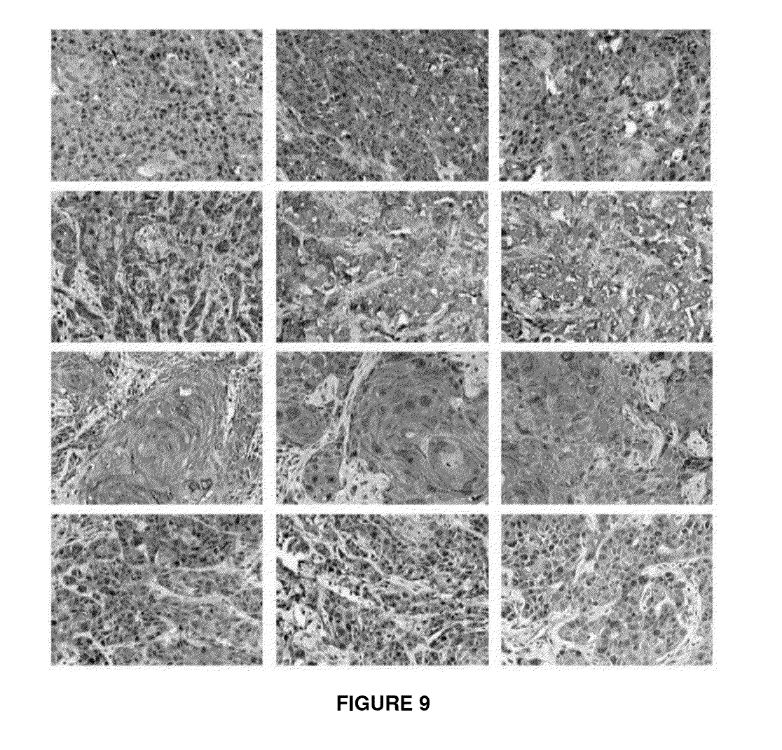

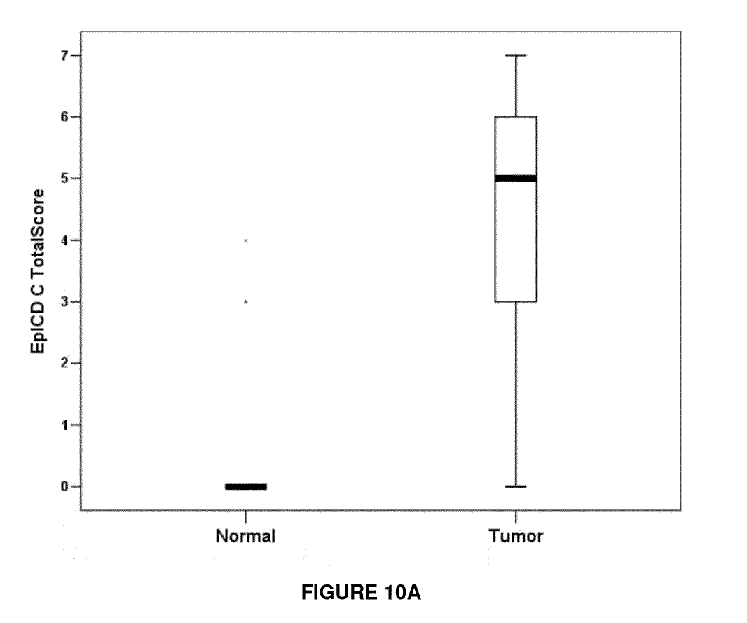

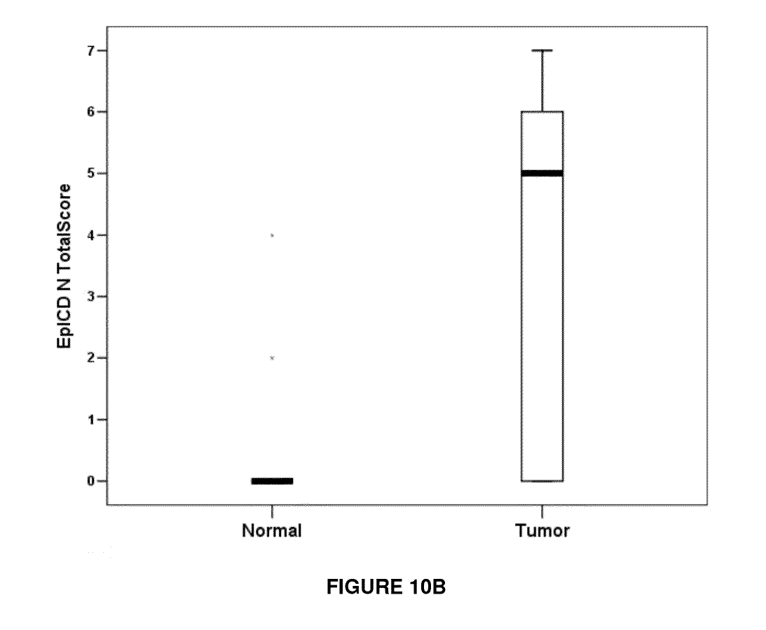

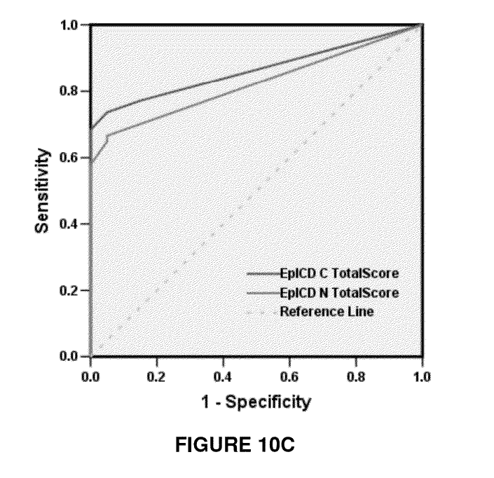

The distribution of Ep-ICD in nuclei of epithelial cells in normal and malignant breast tissues, normal and malignant prostate tissues, and in cancers of lung, colon and rectum, liver, urinary bladder, ovary and pancreas analysed are shown in the bar diagram in FIG. 5. Receiver operating curves were used to determine the sensitivity and specificity of nuclear Ep-ICD in epithelial cancers. In breast cancer, nuclear Ep-ICD showed a sensitivity of 94.12% and specificity of 100%, with area under the curve of 0.968 with an IHC score cutoff value >1.7 (FIG. 6). In prostate cancer, nuclear Ep-ICD showed a sensitivity of 95.56% and specificity of 100%, with area under the curve of 0.973 with an IHC score cutoff >2.67 (FIG. 7). Analysis of Ep-ICD expression in head and neck normal and cancer tissues showed nuclear localization in tumor cells (FIGS. 8 and 9). The results of Ep-ICD, EpEx and beta-catenin expression analysis in head and neck cancer are summarized in Tables 1, 2, and 3 respectively. In head and neck cancer, nuclear Ep-ICD showed a sensitivity of 66.67% and specificity of 95%, with area under the curve of 0.822 with an IHC score cutoff value >1.7 (FIG. 10). Analysis of Ep-ICD expression in esophageal cancer tissues showed nuclear localization in tumor cells (FIG. 11 and Table 4). The nuclear and cytoplasmic distribution of Ep-ICD in epithelial cells in normal and malignant breast tissues, normal and malignant prostate tissues, and in cancers of lung, colon and rectum, liver, urinary bladder, ovary and pancreas analysed are summarized in Table 5. Receiver operating curves analyses were carried and the sensitivity, specificity, area under the curve (AUC) values for nuclear and cytoplasmic distribution of Ep-ICD in cancers of prostate, breast, head and neck and esophagus are summarized in Table 6.

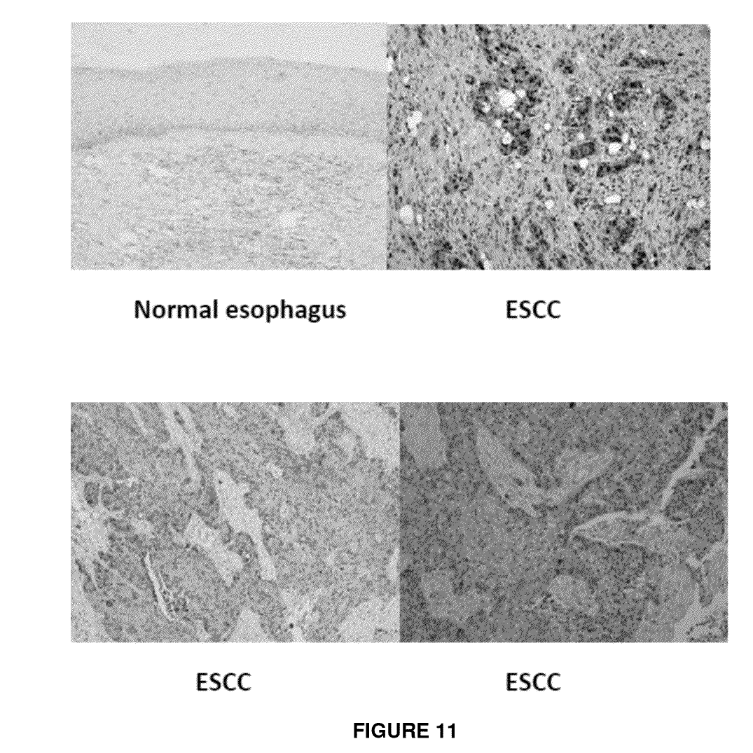

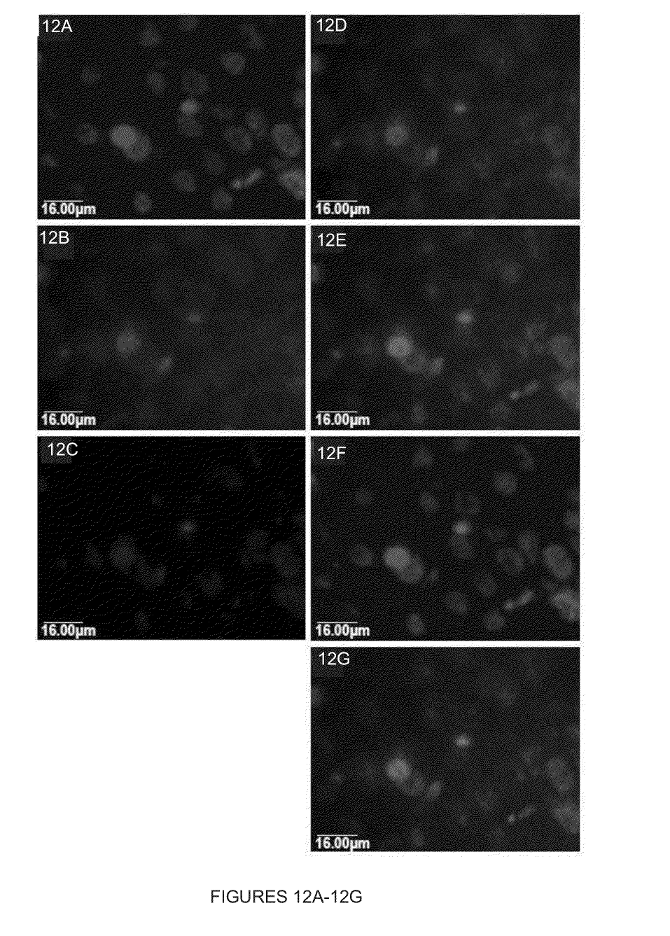

The immunohistochemical data were verified by immunofluorescence analysis. FIG. 12 shows Ep-ICD, EpEx and nuclear DNA subcellular localization in human breast carcinomas. Ep-ICD (red), EpEx (green) and nucleic DNA (blue) were monitored with specific antibodies and DAPI, respectively. A. Nucleic DNA stained with DAPI (blue). B. Subcellular localization of Ep-ICD (red) in breast cancer cells. C. Subcellular localization of EpEx (green) in breast cancer cells. D. Merged image of B&C showing Ep-ICD localized in cytoplasm and nuclei. E. Merged image of A&B showing the nuclear colocalization of Ep-ICD and DAPI. F. Merged image of A&C showing dominating DAPI nuclear staining. G. Merged image of A&B&C reshowing the nuclear colocalization of Ep-ICD and DAPI, also the cytoplasmic Ep-ICD.

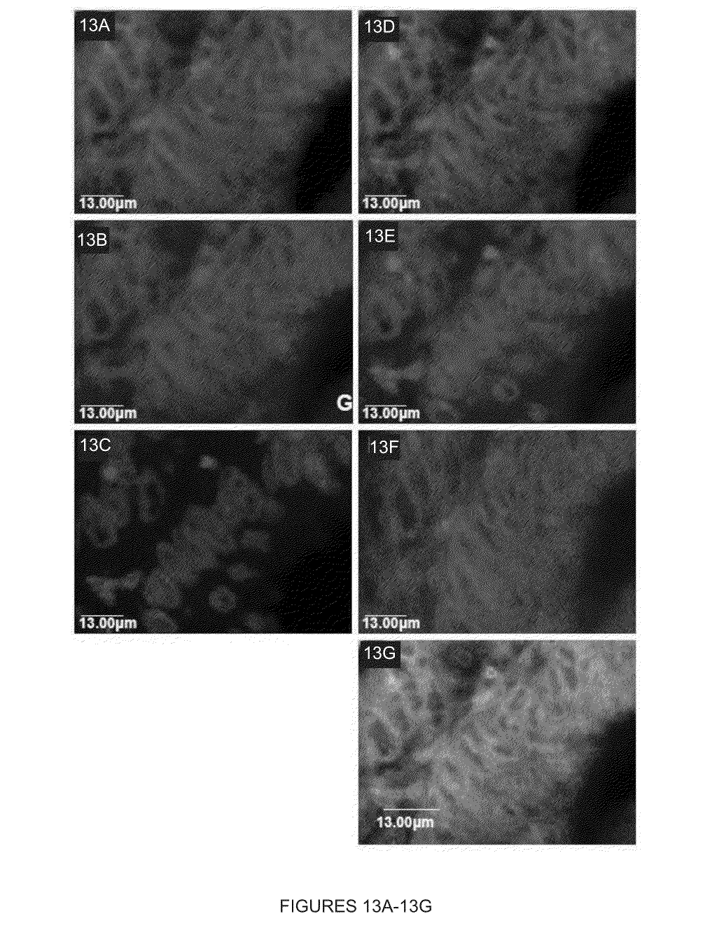

Immunofluorescence analysis of Ep-ICD, EpEx and DNA subcellular localization in human colon carcinomas is shown in FIG. 13. Ep-ICD (red), EpEx (green) and nucleic DNA (blue) were monitored with specific antibodies and DAPI, respectively in colon cancer paraffin section. A. Nucleic DNA stained with DAPI (blue) in colon cancer cells. B. Subcellular localization of Ep-ICD (red) in colon cancer cells. Strong cytoplasmic staining and medium level of nuclear staining of Ep-ICD were demonstrated in colon cancer cells. C. Subcellular localization of EpEx (green) in colon cancer. Strong cytoplasmic staining and low level of membrane staining were displayed in colon cancer cells. D. Merged image of A&C showing dominating DAPI nuclear staining and strong EpEx cytoplasmic staining. E. Merged image of A&B showing the nuclear colocalization of Ep-ICD and DAPI. F. Merged image of B&C showing Ep-ICD colocalized with EpEx in cytoplasm and also appeared in nuclei. G. Merged image of A&B&C showing the nuclear colocalization of Ep-ICD and nuclear DNA, also showing the cytoplasmic Ep-ICD and EpEx in colon cancer cells.

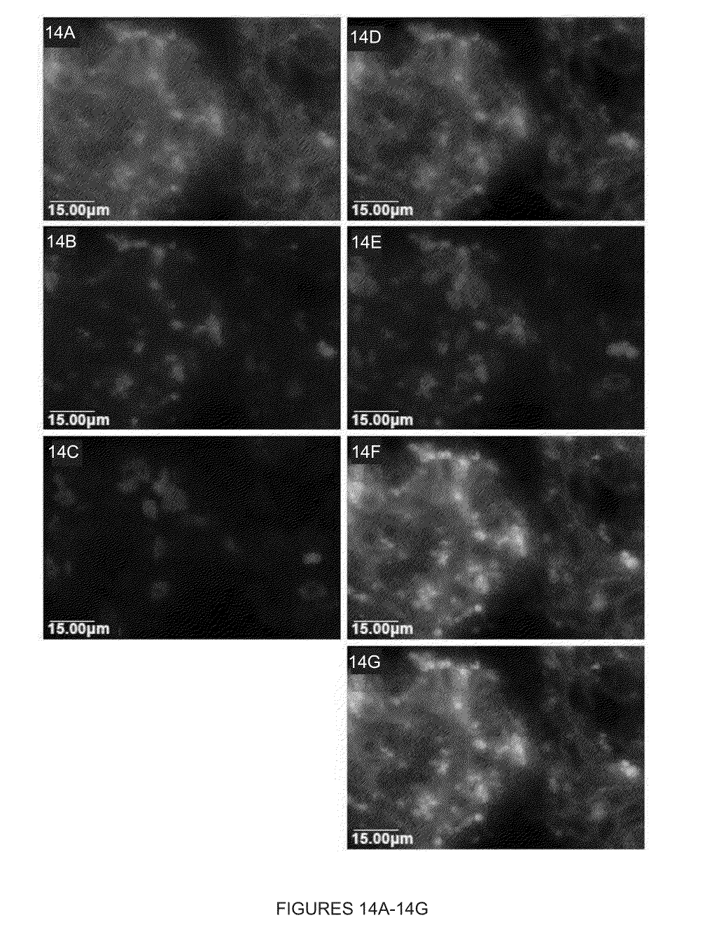

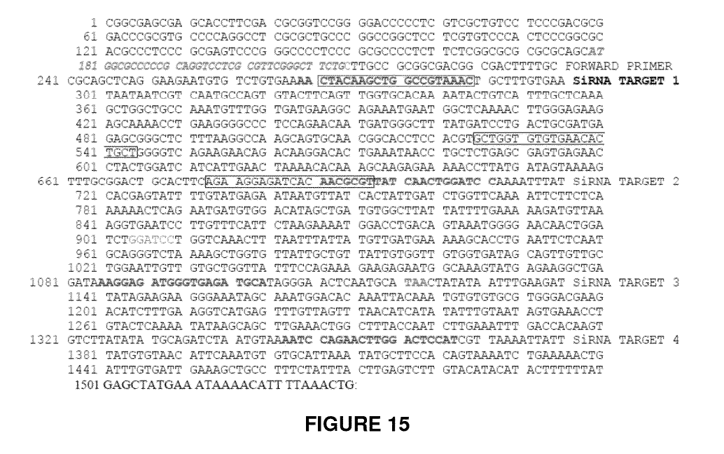

Representative immunofluorescence micrographs of Ep-ICD, EpEx and DNA subcellular localization in human prostate carcinomas is shown in FIG. 14. Subcellular localization of Ep-ICD (red), EpEx (green) and nucleic DNA (blue) were observed with specific antibodies and DAPI, respectively in human prostate cancer paraffin section. A. Nucleic DNA stained with DAPI (blue) in prostate cancer cells. B. Subcellular localization of Ep-ICD (red) in prostate cancer cells. Strong cytoplasmic staining and medium level of nuclear staining of Ep-ICD were demonstrated in prostate cancer cells. C. Subcellular localization of EpEx (green) in prostate cancer cells. Strong cytoplasmic staining and low level of membrane staining were observed. D. Merged image of A&C showing DAPI nuclear DNA staining, weak EpEx membrane staining and strong cytoplasmic staining in prostate cancer. E. Merged image of A&B showing the nuclear colocalization of Ep-ICD and DAPI. F. Merged image of B&C showing Ep-ICD colocalized with EpEx in cytoplasm. G. Merged image of A&B&C showing the nuclear colocalization of Ep-ICD and nuclear DNA, also showing the cytoplasmic Ep-ICD and EpEx in prostate cancer cells.

TABLE 1
Analysis of Ep-ICD protein expression in head and neck cancer

| Clinico- | | Cyto- | | | | | | | |
|---|---|---|---|---|---|---|---|---|---|
| pathological | Total | plasmic | Positivity | p- | OR | Nuclear | Positivity | p- | OR |
| Features | Cases | N | (%) | value | (95% CI) | N | (%) | value | (95% CI) |
| Normal | 20 | 3 | 15.0 | | | 2 | 10 | | |
| HNSCC | 57 | 44 | 77.2 | <0.001 | 28.77 | 39 | 68.4 | <0.001 | 39.0 |
| | | | | | (5.86-141.13) | | | | (4.82-315.21) |

TABLE 2
Analysis of EpEx protein expression in head and neck cancer

| Clinico- | | | |
|---|---|---|---|
| pathological | Total | Cytoplasmic | Positivity |
| Features | Cases | N | (%) |
| Normal | 20 | 0 | |
| HNSCC | 57 | 5 | 8.7 |

TABLE 3
Analysis of B-Cat Protein Expression In Head And Neck Cancer

| Clinico- | | | | | |
|---|---|---|---|---|---|
| pathological | Total | Membranous | Positivity | Cytoplasmic | Positivity |
| Features | Cases | N | (%) | N | (%) |
| Normal | 20 | 1 | 5.0 | 0 | |
| HNSCC | 57 | 3 | 5.2 | 4 | 7.0 |

TABLE 4
Analysis Of Ep-ICD Protein Expression In Esophageal Cancer

| Clinico- | | Only | | Overall |
|---|---|---|---|---|
| pathological | Total | Cytoplasmic | Nuclear | positivity |
| Features | Cases | N % | N(%) | N(%) |
| Normal | 20 | 5(25) | 3(15) | 8(40) |
| ESCC | 46 | 16(35) | 16(35) | 32(70) |

TABLE 5
Immunohistochemical Analysis of Ep-ICD in Normal and
Cancerous Epithelia

| | | | | | Cyto- | Cyto- |
|---|---|---|---|---|---|---|
| | Cancer | Number | Nuclear | Nuclear | plasmic | plasmic |
| Tissue | or | Tissues | Positive | Positivity | Positive | Positivity |
| Type | Normal | (n) | (n) | (%) | (n) | (%) |
| Prostate | Cancer | 49 | 40 | 82 | 40 | 82 |
| | Normal | 9 | 2 | 22 | 1 | 11 |
| | BPH | 21 | 0 | 0 | 1 | 5 |
| Breast | Cancer | 38 | 31 | 82 | 32 | 84 |
| | Normal | 25 | 0 | 0 | 0 | 0 |
| Lung | Cancer | 59 | 47 | 80 | 56 | 95 |
| Colon | Cancer | 59 | 49 | 83 | 46 | 78 |
| Ovarian | Cancer | 10 | 10 | 100 | 10 | 100 |
| Pancreas | Cancer | 10 | 3 | 30 | 2 | 20 |
| Liver | Cancer | 9 | 9 | 100 | 8 | 89 |
| Bladder | Cancer | 10 | 9 | 90 | 9 | 90 |

Note: A cutoff value of 4 was used to determine positivity. BPH, benign prostate hyperplasia.

TABLE 6
Biomarker Analysis of Nuclear and Cytoplasmic Ep-ICD
Expression in Epithelial Cancers

| | | Sensi- | Speci- | | | Asymp- |
|---|---|---|---|---|---|---|
| | | tivity | ficity | PPV | NPV | totic |
| | AUC | (%) | (%) | (%) | (%) | Sig. |
| Ep-ICD Nuclear | | | | | | |
| Staining Scores | | | | | | |
| Prostate Cancer vs. | 0.867 | 82 | 78 | 95 | 44 | 0.001 |
| Normal | | | | | | |
| Breast Cancer vs. | 0.905 | 82 | 100 | 100 | 78 | 0.000 |
| Normal | | | | | | |
| HNSCC vs. Normal | 0.822 | 65 | 95 | 97 | 49 | 0.000 |
| ESCC vs. Normal | 0.630 | 37 | 90 | 90 | 38 | 0.001 |
| Ep-ICD | | | | | | |
| Cytoplasmic Staining | | | | | | |
| Scores | | | | | | |
| Prostate Cancer vs. | 0.880 | 82 | 89 | 98 | 47 | 0.000 |
| Normal | | | | | | |
| Breast Cancer vs. | 0.928 | 84 | 100 | 100 | 81 | 0.000 |
| Normal | | | | | | |
| HNSCC vs. Normal | 0.864 | 74 | 95 | 98 | 56 | 0.000 |
| ESCC vs. Normal | 0.758 | 70 | 70 | 84 | 50 | 0.001 |

### Example 2

### Nuclear Ep-ICD Accumulation can be Used to Predict Aggressive Clinical Course in Early Stage Breast Cancer Patients

Methods

Patient and Tumor Specimens

This retrospective study of biomarkers using the breast cancer patients' tissue blocks stored in the archives of the Department of Pathology and Laboratory Medicine and their anonymized clinical data was approved by the Mount Sinai Hospital Research Ethics Board, Toronto, Canada. The patient cohort consisted of 266 breast cancer patients treated at Mount Sinai Hospital (MSH) between 2000 and 2007. The series consisted of patients who had mastectomy or lumpectomy.

Inclusion criteria: Breast cancer tissue samples of patients that had up to 60 months follow-up with or without an adverse clinical event; availability of clinical, pathological and treatment data in the clinical database.

Exclusion criteria: Breast cancer tissues were not considered for this study if patient follow-up data were not available in the clinical database.

Normal breast tissues were chosen from breast reduction surgeries, normal tissue with adjacent benign lesions, and prophylactic mastectomies. Normal breast tissues from adjacent cancers were not included in this study. The patient cohort consisted of individuals with invasive ductal carcinoma (IDC) (n=180), invasive lobular carcinoma (ILC) (n=15), invasive mucinous carcinoma (IMC) (n=9), ductal carcinoma in situ (DCIS) (n=61), and lobular carcinoma in situ (LCIS) (n=1) and 45 individuals with normal breast tissues. The diagnosis was based on histopathological analysis of the tissue specimens. The follow-up time for all patients including IDC cases in the study was 60 months. The clinicopathological parameters recorded included age at surgery, tumor histotype, tumor size, AJCC pTNM stage, nodal status, tumor grade, recurrence of disease, ER/PR status, hormonal treatment, radiation therapy, and/or chemotherapy. Formalin-fixed paraffin-embedded tissue blocks of all patients included in this study were retrieved from the MSH tumor bank, reviewed by the pathologists and used for cutting tissue sections for immunohistochemical staining with Ep-ICD and EpEx specific antibodies as described below.

Immunohistochemistry (IHC)

Formalin-fixed paraffin embedded sections (4 μm thickness) of breast carcinomas were used for Ep-ICD and EpEx immunostaining as described [Ralhan et al., BMC Cancer 2010, 10(1):331.]. In brief, for EpEx following deparaffinization and rehydration, antigen retrieval was carried out using a microwave oven in 0.01 M citrate buffer, pH 3.0 and endogenous peroxidase activity was blocked by incubating the tissue sections in hydrogen peroxide (0.3%, v/v) for 20 min. For Ep-ICD, the tissue sections were de-paraffinized by baking at 62° C. for 1 hour in vertical orientation, treated with xylene and graded alcohol series, and the non-specific binding was blocked with normal horse or goat serum. Rabbit anti-human Ep-ICD monoclonal antibody from Epitomics Inc. (Burlingame, Calif.) was used in this study. The α-Ep-ICD antibody 1144 recognizes the cytoplasmic domain of human EpCAM and has been used in our previous study of Ep-ICD expression in thyroid carcinoma and other epithelial cancers [Ralhan et al., BMC Cancer 2010]. Anti-EpCAM monoclonal antibody EpEx (MOC-31, AbD Serotec, Oxford, UK) recognizes an extracellular component (EGF1 domain-aa 27-59) in the amino-terminal region [Chaudry et al., Br J Cancer 2007, 96(7):1013-1019]. The sections were incubated with either α-Ep-ICD rabbit monoclonal antibody 1144 (dilution 1:1500) or mouse monoclonal antibody MOC-31 (dilution 1:200) for 60 minutes, followed by biotinylated secondary antibody (goat anti-rabbit or goat anti-mouse) for 20 minutes. The sections were finally incubated with VECTASTAIN Elite ABC Reagent (Vector Laboratories, Burlington, ON, Canada) and diaminobenzidine was used as the chromogen. Tissue sections were then counterstained with hematoxylin. Negative controls comprised of breast tissue sections incubated with isotype specific IgG in place of the primary antibody, and positive controls (colon cancer tissue sections known to express Ep-ICD) were included with each batch of staining for both Ep-ICD and EpEx.

Evaluation of IHC and Scoring

Immunopositive staining was evaluated in the five most pathologically aggressive areas of the tissue sections by two researchers blinded to the final outcome and the average of these five scores was calculated as described by us [[Ralhan et al., BMC Cancer 2010]. Sections were scored on the basis of both the percentage of immunopositive cells and intensity of staining. For percentage positivity, cells were assigned scores based on the following scheme: 0, <10% cells; 1, 10-30% cells; 2, 31-50% cells; 3, 51-70% cells; and 4, >70% cells showing immunoreactivity. Sections were also scored semi-quantitatively on the basis of intensity of staining as follows: 0, none; 1, mild; 2, moderate; and 3, intense. A final score (ranging from 0 to 7) for each tissue section was obtained by adding the scores of percentage positivity and intensity for each of the breast cancer tissue sections. The average total score from the five areas was used for further statistical analysis. Each tissue section was scored for cytoplasmic and nuclear Ep-ICD as well as for membrane EpEx following this scoring scheme.

ELSI Scoring

Following the evaluation and scoring of the IHC data, a calculation was made of the Ep-ICD Subcellular Localization Index (ESLI). The ESLI was calculated according to the following equation: ESLI=½×(% positivity score of Nuclear Ep-ICD+intensity score of Nuclear Ep-ICD+% positivity score of Cytoplasm Ep-ICD+intensity score of Cytoplasm Ep-ICD). As indicated above, the % positivity score comprises a score on a scale of 0 to 4 and the intensity score comprises a score on a scale of 0 to 3.

A cut off of 3 was used for the ESLI score. A significant association was observed between ESLI index positivity and reduced disease-free survival in all breast cancer patients (p <0.001), median survival for ESLI positive cases was 139.3 months and ESLI negative cases was 115.5 months. A significant association was observed between ESLI index positivity and reduced disease-free survival in invasive ductal carcinoma (IDC) patients (p<0.001), median survival for ESLI positive cases was 141.3 months and ESLI negative cases was 115.5 months (p<0.001).

The results from the ESLI scoring is shown in FIGS. 18A and 18B.

Statistical Analysis

The immunohistochemical data were subjected to statistical analysis with SPSS 21.0 software (SPSS, Chicago, Ill.) and GraphPad Prism 6.02 software (GraphPad Software, La Jolla, Calif.) as described previously [Ralhan et al., Mol Cell Proteomics 2008, 7(6):1162-1173]. A two-tailed p-value was obtained in all analyses and a p value <0.05 was considered statistically significant. Chi-square analysis was used to determine the relationship between Ep-ICD and EpEx expression and the clinicopathological parameters. Disease-free survival was analyzed by the Kaplan-Meier method and multivariate Cox regression. Hazard ratios (HR), 95% confidence intervals (95% CI), and p values were estimated using the log-rank test. Disease-free survival or adverse clinical event (defined as clinical recurrence, distal metastases, and/or death) was considered to be the endpoint of the study. The cut-offs for statistical analysis were based upon the optimal sensitivity and specificity obtained from the Receiver operating curves as described before [Ralhan et al., PLoS One 2010, 5(11):e14130]. For nuclear Ep-ICD, an IHC score cut-off value of ≧2 was defined as immunopositive for all tissues analyzed for statistical analysis. Ep-ICD cytoplasmic positivity was considered positive with an IHC cut-off value of ≧4. Membranous EpEx positivity was defined as membrane EpEx IHC score of ≧3.

Results

The clinicopathological parameters and treatment details of 266 breast carcinomas, including 180 IDC cases and 45 normal controls are summarized in Table 7. The median age of patients was 59.9 years (range 30.6-89.8 years). AJCC pTNM Stage I (35.3%) and II (32.7%) comprised a large proportion of tumors in this cohort. Tumor grades distribution was Grade I-21.1%; II-39.8%, and III-32.0%. Among the IDC cases, majority were also AJCC pTNM Stage I (62.8%) and II (32.2%). The IDC cases comprised of Grade I-23.3%; Grade II-36.7%; and Grade III-36.1% tumors.

TABLE 7
Clinicopathologial Characteristics Of Breast
Cancer Patients In The Study Cohort

| | |
|---|---|
| Breast | |
| Cancer | IDC |
| (n = 266) | (n = 180) |

| Surgical Treatment | | | | |
|---|---|---|---|---|
| Lumpectomy | 168 | (63.1%) | 113 | (62.8%) |
| Mastectomy | 84 | (31.6%) | 59 | (32.8%) |
| Unknown | 14 | (5.3%) | 8 | (4.4%) |

Age at diagnosis (years)

| Median (Range - 30.6-89.8) | 59.2 | | 59.2 | |
|---|---|---|---|---|
| <59 yrs | 126 | (47.4%) | 88 | (48.9%) |
| ≧59 yrs | 140 | (52.6) | 92 | (51.1%) |

Adjuvant treatment
Hormonal treatment

| | | | | |
|---|---|---|---|---|
| Tamoxifen | 131 | (49.2%) | 94 | (52.2%) |
| Aromatase Inhibitor | 13 | (4.9%) | 8 | (4.4%) |
| Chemotherapy | 73 | (2.7%) | 66 | (24.8%) |
| Radiotherapy | 149 | (56.0%) | 101 | (56.1%) |
| Therapy details not available | 51 | (19.1%) | 30 | (16.6%) |

| | | |
|---|---|---|
| Tumor size (cm) | | |
| Mean ± SD | 1.85 ± 1.525 | 1.82 ± 1.466 |
| Minimum | 0.1 | 0.1 |
| Maximum | 9 | 9 |
| ≦2 cm | 198 | 81 |
| >2 cm | 57 | 96 |
| Unknown | 11 | 3 |
| AJCC pTNM Stage (n, %) | | |

| 0 (DCIS + LCIS) | 62 | (23.3%) | — | |
|---|---|---|---|---|
| I | 94 | (35.3%) | 113 | (62.8%) |
| II | 87 | (32.7%) | 58 | (32.2%) |
| III | 6 | (2.3%) | 5 | (2.8%) |
| IV | 17 | (6.4%) | 4 | (2.2%) |
| Estrogen receptor (ER) | | | | |
| Negative | 35 | (13.1%) | 33 | (18.3%) |
| Positive | 161 | (60.6%) | 136 | (75.6%) |
| Unknown | 70 | (26.3%) | 11 | (6.1%) |

Progesterone receptor (PR)

| | | | | |
|---|---|---|---|---|
| Negative | 71 | (26.7%) | 64 | (35.6%) |
| Positive | 123 | (46.2%) | 103 | (57.2%) |
| Unknown | 72 | (27.1%) | 13 | (7.2%) |

Grade

| | | | | |
|---|---|---|---|---|
| I | 56 | (21.1%) | 42 | (23.3%) |
| II | 106 | (39.8%) | 66 | (36.7%) |
| III | 85 | (32.0%) | 65 | (36.1%) |
| Unknown | 19 | (7.1%) | 7 | (3.9%) |

Nodal status

| | | | | |
|---|---|---|---|---|
| Negative | 204 | (76.7%) | 123 | (68.3%) |
| Positive | 62 | (23.3%) | 57 | (31.7%) |

Expression of Ep-ICD and EpEx in Breast Cancer Tissues

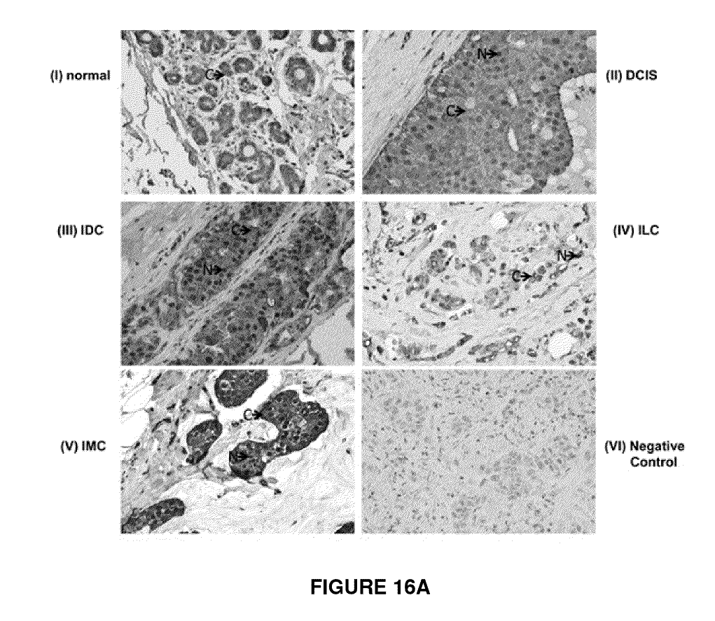

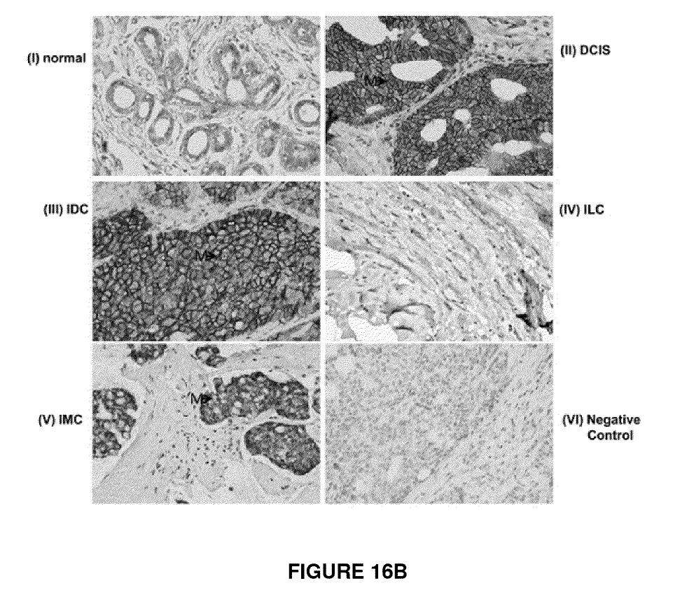

To determine the pattern of expression of Ep-ICD and EpEx in breast cancer histotypes, tissues of DCIS, IDC, ILC, and IMC were analyzed by IHC and compared to normal breast tissues. A summary of the percentage positivity for nuclear Ep-ICD, cytoplasmic Ep-ICD, and membranous EpEx and loss of membranous EpEx is provided in Table 8. Representative photomicrographs of Ep-ICD and EpEx expression in breast cancer subtypes are shown in FIGS. 16(A and B). Of 266 breast carcinomas examined, 121 (46%) were positive for nuclear Ep-ICD and 185 (70%) were positive for membranous EpEx, while 81 cases showed loss of membranous EpEx expression. This compares to 11 of 45 (24%) normal breast tissues immunopositive for nuclear Ep-ICD and 19 of 45 (42%) positive for membranous EpEx. Notably, 12 of 15 (80%) ILCs showed loss of membranous EpEx, compared to 14 of 61 (23%) DCIS, 52 of 180 (29%) IDC, and 3 of 9 (33%) IMC. Cytoplasmic Ep-ICD was frequently present in all histologic subtypes examined and normal tissues (87% normal tissues, 79% DCIS, 81% IDC, 80% ILC, and 100% IMC). Nuclear Ep-ICD was more frequently positive in breast carcinomas (121 of 266, 46%) compared to normal tissues (11 of 45, 24%). Evaluation of the individual subtypes showed nuclear Ep-ICD accumulation was frequently detected in ILC (10 of 15 tumors, 67%), 30 of 61 (49%) DCIS, 75 of 180 (42%) IDC, and 5 of 9 (56%) IMC cases.

TABLE 8
Expression Of Nuclear And Cytoplasmic Ep-ICD And Membranous
Epex In Normal Tissues And Breast Cancer Histotypes.

| | Number | Nuclear | Cytoplasmic | Membranous | Loss of |
|---|---|---|---|---|---|
| | of | Ep-ICD | Ep-ICD | EpEx | membranous |
| | Tissues | Positivity | Positivity | Positivity | EpEx |
| | N | n (%) | n (%) | n (%) | n (%) |
| Tissue type | | | | | |
| Normal | 45 | 11 (24%) | 39 (87%) | 19 (42%) | 26 (58%) |
| Breast Cancer | 266 | 121 (46%) | 215 (81%) | 185 (70%) | 81 (30%) |
| Histotypes* | | | | | |
| DCIS | 61 (22.9%) | 30 (49%) | 48 (79%) | 47 (77%) | 14 (23%) |
| IDC | 180 (67.6%) | 75 (42%) | 145 (81%) | 128 (71%) | 52 (29%) |
| ILC | 15 (5.6%) | 10 (67%) | 12 (80%) | 3 (20%) | 12 (80%) |
| IMC | 9 (3.4%) | 5 (56%) | 9 (100%) | 6 (67%) | 3 (33%) |

For nuclear Ep-ICD a cut off of ≧2 was used to determine positivity. For cytoplasmic Ep-ICD the cut off was a ≧4. For membranous EpEx a cut off of ≧3 was considered positive. *1 LCIS was also included in the study (data not shown in table).

Relationship of Ep-ICD with Clinicopathological Characteristics of IDC Patients.

Nuclear and cytoplasmic Ep-ICD expression in IDC patients and their association with the clinicopathological characteristics are given in Table 9. Notably, nuclear Ep-ICD accumulation was significantly associated with and observed in all IDC patients with clinical recurrences [25 of 25 patients, 100%; p<0.001, Odds ratio (OR)=1.50, 95% confidence interval (CI)=1.28-1.76]. Nuclear Ep-ICD overexpression was significantly associated with early tumor grade (Grade I and II) (53 of 108 patients, 49%; p=0.018, OR=0.46, 95% CI=0.24-0.89) and no lymph node metastases at surgery (58 of 123 patients, 47%; p=0.028, OR=0.48, 95% CI=0.24-0.98). Cytoplasmic Ep-ICD accumulation was also observed in all but one patient with clinical recurrence (24 of 25 patients, 96%; p=0.035, OR=6.75, 95% CI=0.88-51.67). No association was observed between nuclear or cytoplasmic Ep-ICD and ER/PR status, AJCC pTNM stage, T-stage, tumor size, or patient's age at diagnosis (Table 9). Membranous EpEx or loss of membranous EpEx did not show significant correlation with any of the clinico-pathological parameters in this cohort of breast cancer patients (data not shown).

TABLE 9
Nuclear and cytoplasmic Ep-ICD expression in invasive ductal carcinoma
(IDC) and correlation with clinicopathological parameters

| Clinico- | Total | Ep-ICD | | | | Ep-ICD | | | |
|---|---|---|---|---|---|---|---|---|---|
| pathological | Cases | Nuclear | | p- | Odd's ratio | Cytoplasm | | p- | Odd's ratio |
| parameters | (n = 180) | n | (%) | value | (95% C.I.) | n | (%) | value | (95% C.I.) |
| IDC cases | | 75 | 42 | — | — | 145 | 81 | — | — |
| Age | | | | | | | | | |
| <59 yrs | 88 | 39 | 44.3 | | | 74 | 84.1 | | |
| ≧59 yrs | 92 | 36 | 39.1 | 0.480 | 0.80 (0.45-1.45) | 71 | 77.2 | 0.241 | 0.64 (0.30-1.36) |
| Tumor Size^{a} | | | | | | | | | |
| ≦2 cm | 81 | 35 | 43.2 | | | 69 | 85.2 | | |
| >2 cm | 96 | 37 | 38.5 | 0.529 | 0.82 (0.45-1.50) | 73 | 76.0 | 0.128 | 0.55 (0.25-1.20) |
| T-stage | | | | | | | | | |
| T₁ + T₂ | 171 | 71 | 41.5 | | | 138 | 80.7 | | |
| T₃ + T₄ | 9 | 4 | 44.4 | 0.862 | 1.13 (0.30-4.34) | 7 | 77.8 | 0.829 | 0.84 (0.17-4.22) |
| Nodal Status | | | | | | | | | |
| Nₓ₊₀ | 123 | 58 | 47.2 | | | 99 | 80.5 | | |
| N₁₋₃ | 57 | 17 | 29.8 | 0.028 | 0.48 (0.24-0.98) | 46 | 80.7 | 0.973 | 1.02 (0.45-2.24) |
| Stage | | | | | | | | | |
| I + II | 159 | 68 | 42.8 | | | 130 | 81.8 | | |
| III + IV | 21 | 7 | 33.3 | 0.410 | 0.67 (0.26-1.74) | 15 | 71.4 | 0.261 | 0.56 (0.20-1.56) |
| Grade^{b} | | | | | | | | | |
| I + II | 108 | 53 | 49.1 | | | 90 | 83.3 | | |
| III | 65 | 20 | 30.8 | 0.018 | 0.46 (0.24-0.89) | 48 | 73.8 | 0.132 | 0.57 (0.27-1.20) |
| Clinical Recurrence | | | | | | | | | |
| No | 155 | 50 | 32.3 | | | 121 | 78.1 | | |
| Yes | 25 | 25 | 100 | <0.001 | 1.50 (1.28-1.76) | 24 | 96.0 | 0.035 | 6.75 (0.88-51.67) |
| ER/PR status^{c} | | | | | | | | | |
| ER⁺ | 136 | 62 | 45.6 | | | 112 | 82.4 | | |
| ER⁻ | 33 | 12 | 36.4 | 0.338 | 1.47 (0.67-3.22) | 25 | 75.8 | 0.386 | 1.49 (0.60-3.71) |
| PR⁺ | 103 | 49 | 47.6 | | | 88 | 85.4 | | |
| PR⁻ | 64 | 25 | 39.1 | 0.282 | 1.42 (0.75-2.67) | 48 | 75.0 | 0.092 | 1.96 (0.89-4.30) |
| ER⁺PR⁺ | 103 | 49 | 47.6 | | | 88 | 85.4 | | |
| ER⁻PR⁻ | 33 | 12 | 36.4 | 0.260 | 1.59 (0.70-3.56) | 25 | 75.8 | 0.197 | 1.96 (0.89-4.30) |

^{a}Tumor Size was available for 177 IDCs only;
^{b}Tumor Grades were available for 173 IDCs only;
^{c}ER and PR status was available for 169 and 167 IDCs only in our clinical databases.

(a) Tumor Size was available for 177 IDCs only; (b) Tumor Grades were available for 173 IDCs only; (c) ER and PR status was available for 169 and 167 IDCs only in our clinical databases.

Occurrence of an adverse clinical event (recurrence, distal metastases, and/or death) among all breast carcinoma patients was observed in 42 of 121 (34.7%) patients. Subgroup analysis of IDC patients alone that were positive for nuclear Ep-ICD showed an adverse clinical event in 25 of 75 (33.3%) patients. In the entire cohort of breast carcinoma patients, only patients who were positive for nuclear Ep-ICD accumulation had adverse clinical events. Evaluation of all patients who had experienced an adverse clinical event or recurrence showed that of these 42 patients, 37 (88.1%) had early stage tumors (AJCC pTNM Stage I or II), while 5 (11.9%) were Stage III or IV tumors. Among the 25 IDC patients who had adverse clinical events, 21 of 25 (84%) had early stage tumors (AJCC pTNM Stage I and II), while 4 of 25 (16%) were AJCC pTNM Stage III and IV cases.

Prognostic Use of Ep-ICD Expression for Disease-Free Survival

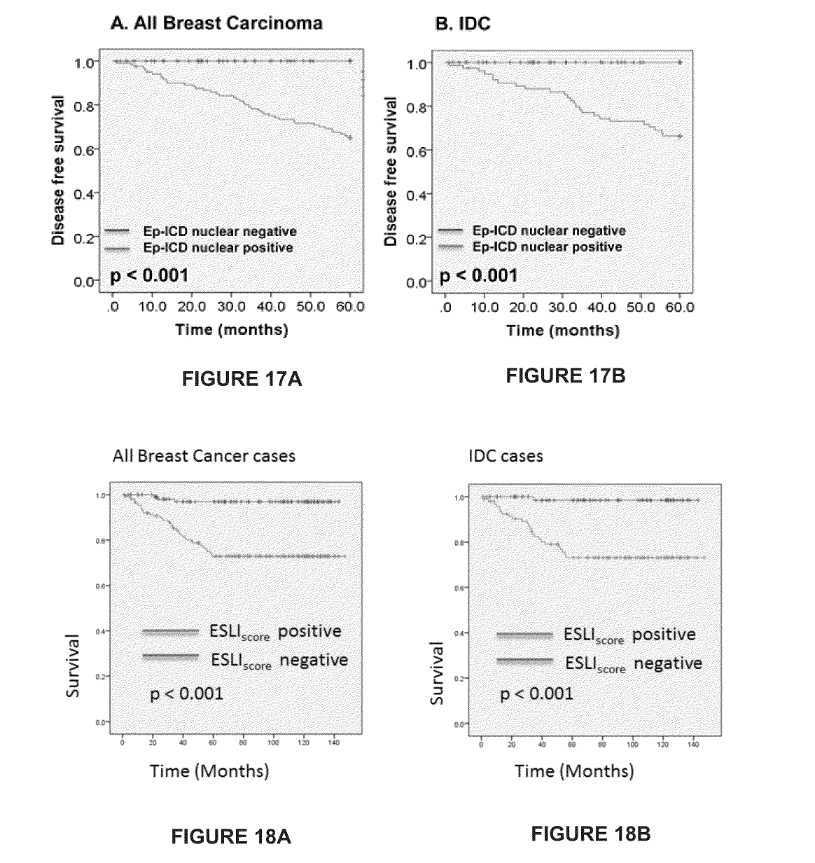

The association between nuclear Ep-ICD accumulation, clinicopathological parameters and disease-free survival was evaluated (Table 10). Significant association was observed between nuclear Ep-ICD expression and disease-free survival (p<0.001), with a decreased third quartile survival time of 40.9 months (FIG. 17A). In contrast, all patients who did not show nuclear Ep-ICD positivity were alive and free of disease even after 5-years post-treatment. Cox multivariate regression analysis identified nuclear Ep-ICD as the most important prognostic marker for an adverse clinical event [p=0.008, Hazard Ratio (HR)=70.47, 95% C.I. 22=3.00-1656.24, Table 10). Subgroup analysis of IDC patients also showed significant association between nuclear Ep-ICD expression and disease-free survival (p<0.001) with a decreased third quartile survival time of 39.5 months (FIG. 17B). In contrast, all patients with no nuclear Ep-ICD positivity were alive and free of disease as of 5-years following surgery. Among the IDC cases, Cox multivariate regression analysis showed nuclear Ep-ICD to be the most important prognostic marker for an adverse clinical event (p=0.011, HR=80.18, 95% C.I. 28=2.73-2352.2). Fifty of the 75 nuclear Ep-ICD positive IDC patients did not have recurrence during this follow up period.

TABLE 10
Kaplan-Meier Survival Analysis And Multivariate Cox
Regression Analysis For Breast Cancer Patients

| | Kaplan-Meier | Multivariate | | |
|---|---|---|---|---|
| | survival | Cox regression | | |
| | analysis | analysis | Hazard's | |
| | unadjusted | adjusted | Ratio | 95% |
| | P-value | P-value | (H.R.) | C.I. |
| All Breast | | | | |
| Carcinomas | | | | |
| Nuclear | <0.001 | 0.008 | 70.47 | 3.00- |
| Ep-ICD⁺ | | | | 1656.24 |
| Cytoplasmic | 0.115 | 0.860 | — | — |
| Ep-ICD⁺ | | | | |
| Age | 0.081 | 0.178 | — | — |
| Tumor size | 0.676 | 0.518 | — | — |
| T-stage | 0.315 | 0.388 | — | — |
| Nodal status | 0.963 | 0.190 | — | — |
| Clinical Stage | 0.064 | 0.260 | — | — |
| Grade | 0.094 | 0.035 | — | — |
| ER status | 0.292 | 0.654 | — | — |
| PR status | 0.827 | 0.790 | — | — |
| IDC Tumors | | | | |
| Nuclear | <0.001 | 0.011 | 80.183 | 2.733- |
| Ep-ICD⁺ | | | | 2352.2 |
| Cytoplasmic | 0.048 | 0.496 | — | — |
| Ep-ICD⁺ | | | | |
| Age | 0.796 | 0.787 | — | — |
| Tumor size | 0.556 | 0.516 | — | — |
| T-stage | 0.237 | 0.366 | — | — |
| Nodal status | 0.814 | 0.398 | — | — |
| Clinical Stage | 0.129 | 0.809 | — | — |
| Grade | 0.329 | 0.062 | — | — |
| ER status | 0.384 | 0.678 | — | — |
| PR status | 0.984 | 0.499 | — | — |

Thus, as indicated above, nuclear Ep-ICD was more frequently expressed in breast cancers as compared to normal tissues. Significant association was observed between increased nuclear Ep-ICD expression and reduced disease-free survival in patients with ductal carcinoma in situ (DCIS) and invasive ductal carcinoma (IDC) (p<0.001). Nuclear Ep-ICD was positive in all the 13 DCIS and 25 IDC patients who had reduced disease-free survival, while none of the nuclear Ep-ICD negative DCIS or IDC patients had recurrence during the follow up period. Notably, majority of IDC patients who had recurrence had early stage tumors. Multivariate Cox regression analysis identified nuclear Ep-ICD as the most significant predictive factor for reduced disease-free survival in IDC patients (p=0.011, Hazard ratio=80.18).

Discussion

Ever since the regulated intramembrane proteolysis of EpCAM was described as a novel mechanism of triggering oncogenic signalling by Maetzel et al (Nat Cell Biol (2009), 11(2):162-171), investigation of Ep-ICD expression in human epithelial cancers for determination of its clinical relevance is in hot pursuit. Our earlier preliminary study reported frequent nuclear and cytoplasmic Ep-ICD expression in ten different epithelial cancers, including a small number of breast cancers (Ralhan et al., BMC Cancer 2010). This first report did not examine the correlation of nuclear Ep-ICD expression with clinical parameters or its prognostic utility in these cancers. The current study assessed the potential suitability of Ep-ICD as a marker in predicting clinical course and aggressiveness of breast cancer. Although expression of the full length EpCAM protein has been widely investigated in human malignancies, the expression and subcellular localization of its intracellular domain Ep-ICD has not been well characterized in clinical specimens. Our study demonstrated differences in expression of Ep-ICD and EpEx between normal and malignant breast tissues and their relationship with disease prognosis, providing valuable information as to their suitability as potential biological markers. Given the interest in the therapeutic potential of EpCAM targeted therapies in cancer management and the limited understanding of the role and expression pattern of Ep-ICD in breast cancer, our study helps to shed light on this widely-studied, yet not fully understood protein. Furthermore, our study is the first in-depth characterization of Ep-ICD expression in IDC of the breast.

Importantly, increased detection of nuclear Ep-ICD in breast carcinomas compared to normal tissues warrants exploration of its potential role in tumorigenesis. The increased regulated intramembrane proteolysis of EpCAM resulting in release of its cytoplasmic domain, Ep-ICD, and its subsequent translocation to the nucleus has been demonstrated to trigger oncogenic signalling in colon carcinoma (Maetzel et al., 2009). In an earlier study, we reported that nuclear Ep-ICD accumulation predicted poor prognosis in thyroid carcinomas and was elevated in patients with anaplastic tumors (Ralhan et al., BMC Cancer 2010). Taken together with our present study, these reports underscore the biological significance of increased nuclear Ep-ICD in cancer. The discovery of the tumor-suppressive properties of EpCAM in some cancers has surprised many researchers, given its association with poor prognosis in many other cancers. Some studies have suggested the tumor microenvironment may be an important factor in dictating whether EpCAM will promote or inhibit tumor progression, particularly given its ability to mediate homophilic adhesive interactions between cells (van der Gun B T F et al., Carcinogenesis, 2010, 31(11):1913-1921). Furthermore, regulated intramembrane proteolysis of EpCAM and the associated oncogenic signalling by Ep-ICD may shed light on some of these observations as additional protein-protein interactions are uncovered (Munz M. et al., Cancer Res 2009, 69(14):5627-5629) and Carpenter G. et al., Cancer Cell 2009, 15(3):165-166). Recently, the endoplasmic reticulum aminopeptidase 2 (ERAP2), a proteolytic enzyme set in the endoplasmic reticulum (ER) has been shown to co-localize with EpCAM in the cytoplasm/ER where it plays a central role in the trimming of peptides for presentation by MHC class I molecules. This association between EpCAM and ERAP2 suggests a new mechanism of EpCAM processing and regulation of antigen presentation in breast cancer (Gadalla S E et al., Biochem Biophys Res Commun 2013, 439(2):203-208).

Our study revealed several important findings with potentially significant implications for the use of Ep-ICD as a biomarker. We observed high occurrence of recurrence, distal metastases, or death among IDC patients who were positive for nuclear Ep-ICD accumulation. In contrast, no recurrence distal metastases, or death were observed in nuclear Ep-ICD negative patients during the follow up period. Importantly, a great majority of patients with recurrence (37 of 42, 88.1%) had early stage breast carcinomas (AJCC pTNM Stage I and II) that would normally be considered lower-risk for future recurrence. Moreover, the fact that only nuclear Ep-ICD positive patients had recurrence and that no nuclear Ep-ICD negative patient suffered the same suggests a potential clinical application for this biomarker. These observations support the notion that nuclear Ep-ICD accumulation even in early stage breast tumors holds promise for predicting aggressive disease.

Indeed, the presence of nuclear Ep-ICD, irrespective of tumor stage or any other clinical variable predicted a high risk of disease recurrence. Multivariate Cox regression analyses identified nuclear Ep-ICD accumulation as the most significant factor for prediction of recurrence in IDC patients. These findings, of course, require further clinical validation in larger number of patients followed prospectively, but are nonetheless encouraging because it may provide a path to identify patients who may require more aggressive monitoring and/or treatment, particularly in patients early stage tumors who show nuclear Ep-ICD accumulation.

The in vitro studies on functional role of EpCAM in breast cancer cell lines demonstrated that transfection of EpCAM resulted in increased nuclear accumulation of β-catenin in MDA-MB-231^{EpCAM} and upregulated Wnt reporter assay activity in Hs578T^{EpCAM} cells suggesting activation of Wnt pathway (Gostner J M et al., BMC Cancer 2011, 11:45). Moreover, the interaction between membranous EpEx and the extracellular environment and nuclear Ep-ICD and intracellular signalling continues to reveal interesting associations. Martowicz et al. (BMC Cancer 2012, 12:501) and others recently reported that cancer cells of an epithelial but not mesenchymal phenotype require EpCAM as an invasion-promoting factor (Sankpal et al., Cancer Res 2009, 6993):753-757) It is possible that nuclear Ep-ICD accumulation is an early indicator of tumor progression, as evidenced by its correlation with lower grade, but also, disease recurrence. Furthermore, the expression of nuclear Ep-ICD and membranous EpEx may have not only prognostic but also therapeutic implications to stratify patients who are likely to respond to EpCAM based immunotherapies. In this context, a recent study in 1365 breast cancers reported EpCAM expression varies significantly and is differentially associated with prognosis in the luminal B HER2 positive, basal like, and HER2 intrinsic subtypes of breast cancer. However, a limitation of this study is the expression of Ep-ICD has not been analysed and only EpCAM expression was correlated with disease outcome. The prevalence of the full length EpCAM and Ep-ICD in a variety of human cancers has been recently reported using tissue microarrays suggesting loss of membranous EpEx is a common event in human epithelial cancers and the ratio of EpEx and Ep-ICD is dependent on the tumor (Fong D. et al., J Clin Pathol 2014, 67(5):408-414). However, this study does not address the clinical relevance of relative expression of EpEx and Ep-ICD in these cancers. Future studies evaluating the prognostic and predictive role of these variants in human cancers, especially in patients treated with Ep-CAM specific antibodies are warranted.

One limitation of our study is that while all the 25 IDC patients that had recurrence were nuclear Ep-ICD positive suggesting nuclear Ep-ICD positivity is a risk factor for aggressive disease in these patients, there were 50 of 75 nuclear Ep-ICD positive IDC patients who did not experience any recurrence during this follow up period. Hence there is a need to identify other protective factors in these patients that prevent the recurrence of disease. Another limitation is the very small number of ILC and IMC cases analyzed in this study. Future studies will be directed to search for additional factors which promote or protect against recurrence in this subgroup of nuclear Ep-ICD positive patients. Nevertheless, our findings are important in stratifying aggressive early stage breast cancer patients who will need rigorous follow-up for more effective disease management. At the same time the absence of nuclear Ep-ICD in early stage breast cancer patients also has the potential to help avoid over-treatment, sparing these patients the harmful side effects of aggressive therapies and reducing health care costs upon validation in future studies.

### CONCLUSIONS

Patients with nuclear Ep-ICD positive breast cancers had poor prognosis. The high recurrence of disease in nuclear Ep-ICD positive patients, especially those with early tumor stage suggests that nuclear Ep-ICD accumulation holds the promise of identifying early stage patients with aggressive disease who are likely to be in need of more rigorous post-operative surveillance and/or treatment. Moreover, the ESLI score developed by the present inventors provides a unique means of arriving at the subject diagnosis. The ESLI scoring method involves a unique combination of quantitative and qualitative values, which provide a simple and useful aid for clinical diagnoses.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the antibodies, methodologies etc. which are reported therein which might be used in connection with the invention. Nothing herein is to be construed as an admission that the invention is not entitled to antedate such disclosure by virtue of prior invention.

We claim:

1. A method for prognosing breast cancer in a subject, the method comprising:
(a) detecting a level of nuclear Ep-ICD in a biological sample from the subject;
(b) comparing the level detected in the biological sample to a control; and
(c) prognosing breast cancer based on the comparison between the detected level of nuclear Ep-ICD and the control.

2. The method of claim 1, wherein:
i) if the control is a level of nuclear Ep-ICD in a non-cancerous biological sample, then a higher detected level of nuclear Ep-ICD indicates a poor prognosis, and an equal or lower detected level of nuclear Ep-ICD indicates a favorable prognosis; or
ii) if the control is the level of nuclear Ep-ICD in a biological sample known not to progress to breast cancer for at least 40 months following measurement of the control level, then a higher detected level of nuclear Ep-ICD indicates a poor prognosis, and an equal or lower detected level of nuclear Ep-ICD indicates a favorable prognosis; or
iii) if the control is the level of nuclear Ep-ICD in a biological sample known to progress to breast cancer in less than about five years following measurement of the control level, then an equal or higher detected level of nuclear Ep-ICD indicates a poor prognosis, and a lower detected level of nuclear Ep-ICD indicates a favorable prognosis.

3. The method of claim 2, wherein a poor prognosis comprises one or more of disease free survival of less than five years and overall survival of less than five years.

4. The method of claim 3, wherein disease free survival is less than or equal to about 41 months.

5. The method of claim 2, wherein a favorable prognosis comprises one or more of disease free survival of at least about five years and overall survival of at least about five years.

6. The method of claim 1, wherein the sample comprises breast epithelial cells.

7. The method of claim 1, wherein the sample comprises breast tissue.

8. The method of claim 1, wherein the sample comprises stage I or II breast cancer tumor cells.

9. The method of claim 1, wherein the breast cancer is invasive ductal carcinoma, invasive lobular carcinoma, invasive mucinous carcinoma, ductal carcinoma in situ or lobular carcinoma in situ.

11. A method for determining whether a subject has breast cancer with a poor prognosis, the method comprising:
detecting the presence or absence of nuclear Ep-ICD in a biological sample from the subject, the biological sample comprising breast epithelial cells, the presence of nuclear Ep-ICD indicating that the subject has breast cancer associated with a poor prognosis.

12. A method for the prognosis of survival time of a subject having breast cancer, the method comprising:
a) detecting the level of nuclear Ep-ICD in a biological sample from the patient;
b) comparing the detected level of nuclear Ep-ICD with one or more predetermined reference values correlated with a specific prognoses of survival time, and
c) determining a prognosis for the subject, wherein a favorable prognosis of survival time is provided for the subject when the detected level of nuclear Ep-ICD is lower than said corresponding predetermined reference values, and wherein a poor prognosis of survival time is provided for the subject when the detected value is lower than said corresponding predetermined cut-off reference values.

13. The method of claim 1, wherein the method includes the step of calculating an Ep-ICD Subcellular Localization Index (ESLI) score.

14. The method of claim 11, wherein the method includes the step of calculating an Ep-ICD Subcellular Localization Index (ESLI) score.

15. The method of claim 12, wherein the method includes the step of calculating an Ep-ICD Subcellular Localization Index (ESLI) score.
